(19) United States
(12) Patent Application Publication
(10) Pub. No.: US 2025/0221809 A1
(43) Pub. Date: Jul. 10, 2025

(54) METHODS AND DEVICES FOR RENAL ACCESS AND PRESSURE REDUCTION

(71) Applicant: ExploraMed V, LLC, Mountain View, CA (US)

(72) Inventors: John Chang, Los Altos, CA (US); Joshua Makower, Los Altos Hills, CA (US); Earl Bright, II, Sunnyvale, CA (US); Theodore Lamson, Alamo, CA (US); Jonathan Podmore, San Carlos, CA (US); Eric Taylor, West Harford, CT (US); Fred Co, Santa Clara, CA (US); John Hanley, Manhattan Beach, CA (US)

(21) Appl. No.: 19/009,753

(22) Filed: Jan. 3, 2025

Related U.S. Application Data
(60) Provisional application No. 63/617,490, filed on Jan. 4, 2024.
(60) Provisional application No. 63/557,156, filed on Feb. 23, 2024.

(51) Int. Cl. A61F 2/04; A61B 17/32; A61B 18/00

(52) U.S. Cl. CPC A61F 2/04; A61B 18/00; A61B 17/320016; A61B 2018/00511; A61F 2002/048; A61F 2220/0016

(57) ABSTRACT

Systems and methods for renal access and pressure reduction. In one or more embodiments, there are provided various systems and methods to reduce renal pressures. In one or more aspects, there are provided devices and methods for the removal or reduction of a capsular constriction to reduce renal pressures such as by one or more of removing fluid or tissue.

### FIELD OF THE DISCLOSURE

The present disclosure generally relates to methods and devices for renal anatomy and capsule access and pressure reduction.

### BACKGROUND OF THE DISCLOSURE

There is a continuing need for safe and effective approaches to treating patients with Heart Failure (HF) and other cardiac and kidney related conditions such as Acute Decompensated Heart Failure (ADHF), Cardiorenal Syndrome (CRS), Acute Kidney Injury (AKI), Chronic Kidney Disease (CKD), Hypertension and Diabetes.

Heart failure is a serious and costly condition that is increasingly prevalent and burdensome to the health care system. In 2014, there were approximately one million hospitalizations with primary HF in the United States. In the same year, there were over three million additional hospitalizations where HF was a comorbidity.

Congestion is the main reason for hospitalization of patients with ADHF with congestion defined as fluid accumulation in the intravascular compartment and the interstitial space. As a result, relief of congestion is the primary therapeutic goal for these patients.

Renal dysfunction is one of the most common and most important comorbidities of heart failure. Several studies have shown that heart failure outcomes are closely related with renal dysfunction. Cardiac and renal diseases interact in a complex, bidirectional and interdependent manner. The term Cardiorenal Syndrome (CRS) has been defined as any acute or chronic problem in the heart or kidneys that could result in an acute or chronic problem of the other.

The kidneys manage salt and fluid balance for the body and as a result their function or dysfunction play a central role in the presence of congestion in the body.

In the patient with heart failure, decreased renal function can lead to decreased GFR, urine output and sodium excretion resulting in activation of the Renin-Angiotensin-Aldosterone System (RAAS) which in turn leads to further salt and water retention and additional fluid overload in an ongoing cycle that impacts patient outcomes.

One mechanism that may lead to renal dysfunction is increased parenchymal pressure leading to the compression of renal structures (such as intrarenal veins, tubules and glomeruli), reduction of renal blood flow and a decrease in Glomerular Filtration Rate (GFR). Increased parenchymal pressure may also inhibit the effectiveness of diuretics which are a key tool in treating congestion for patients with ADHF by impacting delivery of the diuretics to their sites of action on the renal tubules.

The kidneys are located in the retroperitoneal space. Each kidney is surrounded by a fibrous collagen capsule that does not stretch appreciably. Pressures up to 10,000 mm HG are required to stretch or rupture the capsule.

Increased renal parenchymal pressure can result from increased volume within the kidney and capsule such as from increased interstitial fluid. Increased renal interstitial pressure may also result from external pressure on the kidney such as from increased intra-abdominal pressure or increased perirenal pressure.

There may be multiple modes by which intracapsular volume and pressure may increase. The parenchymal volume and pressure may increase due to renal congestion or the presence of a hematoma such as the Page Kidney. Increased fluid volume may also lead to increased pressure-such as increased urine/fluid in the collecting chambers (pelvis, calyces) due to ureteral blockage, increased fluid/lymph in between the parenchyma and capsule due to ‘weeping’ or other mechanisms of fluid escape. In all cases, the volume increase inside the renal capsule is eventually constrained by the non-compliant membrane and once constrained, the parenchymal pressures will increase rapidly with additional volume.

Intra-abdominal pressure may increase due to ascites or increased fluid in the splanchnic system, which may then apply pressure onto the renal system. Patients with morbid obesity may also experience increase intrarenal pressure.

Moreover, Perirenal pressure may occur with increased adipose tissue within the perirenal fascia—leading to compression and increased pressure on the kidney.

Elevated interstitial space pressure can compress and compromise glomeruli, renal veins, and perhaps more so, renal tubules. This phenomenon is specifically acute within the kidney because it is encapsulated by the fibrous capsule that does not allow sufficient expansion of the congested organ. The renal lymphatic system is the means by which interstitial fluid is cleared from the kidney. With increased renal venous pressure the lymphatic system can become overloaded. Increased interstitial pressure paradoxically can collapse lymphatic vessels, further reducing drainage. Additionally, elevated central venous pressure reduces or even reverses the pressure differential of the lymphatic system, reducing drainage in the subclavian veins, causing tissue congestion and insufficient lymphatic drainage. Moreover, in a turgid kidney, with a constraining renal capsule, the lymphatic vessels that permeate the capsule can become constricted.

Compression of the perirenal space can lead to activation of the Renin Angiotensin Aldosterone System that hormonally reduces renal perfusion and increases renal venous pressure, potentially leading to further intrarenal congestion. Certain procedural interventions may have a less positive effect over time if it causes adipose tissue to active the RAAS. Additionally, congestive heart failure can lead to ascites and splanchnic reservoir filling, both contributing to increase abdominal pressure. It has been further noted that a reliably low-pressure system in congestive heart failure is in the collecting duct calyces of the kidney and upper urinary tract emptying in the bladder. Accordingly, the lymphatic system can be employed to alleviate elevated intrarenal interstitial pressures.

Removal or reducing that capsular constriction can reduce renal pressures significantly and improve renal function. Additionally, releasing/reducing/relieving renal pressure in a minimally invasive manner may have immense benefits for patients with cardiac and kidney related conditions such as ADHF, CKD, AKI, Diabetes, hydronephrosis and hypertension.

Accordingly, there is a need for apparatus and methods that provide for renal capsule access, the establishment of an alternative drainage pathway for interstitial fluid and pressure reduction within the renal capsule and/or kidney. There is also a need for apparatus and methods that anticipate or accommodate the changes in the renal system over time.

The present disclosure addresses these and other needs.

### SUMMARY OF THE DISCLOSURE

Briefly and in general terms, the present disclosure is directed towards systems, devices and methods for renal anatomy access and pressure reduction. In one or more aspects, there are provided devices and methods for the removal or reduction of a capsular constriction to reduce renal pressures. Reducing renal interstitial pressure can be accomplished by one or more of removing fluid or tissue or by removing portions or an entirety of containing tissues which allows renal tissues to expand or relax in a larger created space.

In one embodiment, there are provided devices and methods to address parenchyma weeping or fluid leakage into an intracapsular space. In various aspects, fluid is drained out of the body, into a perirenal space, a peritoneal space and/or back into the natural drainage pathway of urine from the kidney. Various apparatus and systems are provided to assess a condition of a kidney and/or provide information concerning a path to engaging the kidney for interventional purposes.

In another embodiment, there are provided devices and methods to reduce pressures related to renal parenchyma swelling or interstitial tissue congestion in a constrained capsular space. In various aspects, tissue surrounding the kidney is modified by one or more of decapsulation, tissue release (linear or non-linear), perforation, scoring or tissue weakening. Various approaches include energy or mechanical means for engaging target tissues and structures.

In certain aspects, devices and methods are employed to remove or reduce renal capsular constriction to significantly reduce renal interstitial pressures. Less than an entirety of the capsule can be removed and/or the capsule can be interrupted via incisions or releases. Moreover, the capsule can alternatively or additionally be enlarged to allow the parenchyma to expand resulting in lowering its internal pressures.

In various embodiments, approaches to target tissues or areas include one or more of catheter approaches involving one or more of the arterial or venous systems, approaches involving the urethra and or ureters, and percutaneous approaches. Visualization and navigation can be provided by fluoroscopy, MRI, ultrasound, direct visualization, endoscopy or the like.

In one or more aspects, there is provided an apparatus for accessing and penetrating a tissue separating the parenchyma of the kidney from other structures and a device, system or means for placing in fluid communication with the parenchyma of the kidney one or more drainage structures, where the drainage structure or structures are dimensioned to permit the flow of fluid from the parenchyma of the kidney into a space outside the parenchyma of the kidney, thus reducing the interstitial pressure. In particular, the capsule can be penetrated from an external approach as well as through a vessel or the uroendothelium internally. Such device, system or means can be anything that is in fluid communication with target tissues, which can be a device inserted only subcapsularly or could be a device or apparatus fully or partially inserted in the parenchyma itself. Drainage can be via the calyx and/or ureter, directly into the perirenal, peritoneal or retro peritoneal space, into a collecting bag with a port or directly out of the body to a collecting system outside the body.

Certain of the approaches to reducing renal pressures can alternatively or additionally involve relatively avascular regions of the kidney such as the Brodel's line, which is a less vascular segment dividing the kidney with two thirds of kidney tissue anterior and one third posterior to the segment. This region can be targeted as puncture sites for accessing a renal capsule to minimize the risk of bleeding.

In one or more embodiments, the capsule can be cut to reduce subcapsular pressure. Cutting of the capsule can be done by approaching from outside of the capsule. In this regard, by controlling the capsule, pulling on the capsule, or injecting liquid through the capsule to dissect the capsule from the parenchyma surface, then cutting the capsule allows the less constrained parenchyma to drain and/or expand. Further, applying tension to an outside of the capsule alone can accomplish the desired dissection of the capsule from the parenchyma.

In various embodiments, temporary or permanent implants can be employed for the removal or reduction of a capsular constriction to reduce renal interstitial pressures. Further, various approaches to address potential regrowth of a capsule are provided including means to maintain patency and/or prevent the growth of a new capsule. In additional aspects, approaches to hemostasis are provided.

In one or more embodiments, multiple points of access or drainage apparatus are employed to reduce renal pressure. The target renal anatomy can present a connected and integrally related interstitial pressure environment or can present multiple compartmentalized interstitial pressure environments. Multiple points of access and/or drainage apparatus can be used to address elevated renal pressures where either connected and integrally related interstitial pressure environments or multiple compartmentalized environments are presented.

Where there is a compartmentalization of interstitial pressure environments, each compartmentalized section of the target renal anatomy can be treated individually or as a group or sub-groups with complementary methods and devices to relieve renal pressures. Accordingly, in one embodiment, an interventional procedure can involve directly treating or relieving the pressure of one or more parenchyma sections of a kidney or additionally or alternatively, the areas between one or more calyx or the calyxes themselves.

In one or more approaches, a stent structure is implanted permanently or temporarily to facilitate reducing fluid build-up and/or pressures existing in renal tissues. The stent can generally form a tubular structure and be defined by a pattern of lattices or woven structure and can further include drug eluting structure or functionality to facilitate healing or prevent or reduce tissue re-growth. The stents may be self-expanding or balloon expandable and placement may be facilitated by pre/post or interim dilation.

In additional or alternative aspects, one or more grommets or tubes can be positioned across a renal capsule to provide a path or channel for reducing renal pressures. In one embodiment, the grommet can include spikes or barbs for attaching the grommet in place. The grommet or tube can further include structure that accomplishes tenting the capsule away from the parenchyma. In this way, an operating space is provided and/or a cavity is created to expel excess fluid from renal tissues. Such grommets or other implanted structures or treatment apparatus can include electronic components that employ telemetry to communicate information concerning the environment in which the apparatus are being deployed or have been implanted. In this way, treatments can be remotely initiated and/or controlled such as by generating energy or releasing therapeutics to further treat renal tissues or inhibit re-growth. The subcapsular boundary between the parenchyma and renal capsule can additionally or alternatively be insufflated to create a desired cavity for relieving renal pressure or creating an operating space. In this regard, a device incorporating a needle that can alternate between sharp and blunt in a reversible or pre-determined non-reversible fashion can be used to puncture and access the capsule.

An interventional device can embody decapsulation structure that completely removes a capsule or alternatively scores or fenestrates a capsule for the purpose of reducing renal pressures. Mechanical and/or energy can be employed to decapsulate the kidney. In one aspect, cautery energy is used to cut tissues and to also prevent or reduce re-growth of capsule or other renal tissues. In one particular or alternative aspect, an ablation device can be employed to weaken or dissect a capsule or can be otherwise used to facilitate drainage. Hemotherapy or chemotherapies can additionally be incorporated into a treatment device to prevent or reduce re-growth. In one particular embodiment, an interventional device includes a sharpened fin or blade that is configured to be dragged or advanced along capsular tissue to accomplish decapsulation. The sharpened fin or other cutting structure can be movable or configured to be retracted when not in use or engaged.

In further additional or alternative aspects, the interventional procedure to reduce renal pressures can involve structure or apparatus that accomplishes gentle massaging or squeezing of renal tissues. In one approach, a jacket can be placed over the kidney and be configured to gently compress the kidney or portions thereof. With drainage or other structures in place, the gentle compression functions to facilitate removing fluids from renal anatomy. In another approach, an expandable member such as a highly flexible balloon can be inserted within a subcapsular location and expanded to gently massage or compress the kidney to facilitate fluid removal.

These and other features of the disclosure will become apparent to those persons skilled in the art upon reading the details of the systems and methods as more fully described below.

### BRIEF DESCRIPTION OF THE DRAWINGS

FIGS. 1A-N are cross-sectional and partial cross-sectional views, depicting approaches to reducing renal pressure.

FIG. 2 is a cross-sectional view, depicting an interventional procedure involving multiple portions of a kidney.

FIGS. 3A-C are cross-sectional and partial cross-sectional views, depicting apparatus for accessing a subcapsular location.

FIGS. 4A-E are cross-sectional and side views, depicting approaches to capsular release.

FIGS. 5A-D are cross-sectional views, depicting further approaches to capsular release.

FIGS. 6A-B are top views, depicting an assembly configured to dissect renal tissue.

FIGS. 7A-M are cross-sectional views, depicting approaches to treating a kidney involving an implant.

FIGS. 8A-D are cross-sectional views, depicting approaches to dissecting capsular tissue.

FIGS. 9A-D are cross-sectional views, depicting an approach to separating a capsule from renal tissues.

FIGS. 10A-B are cross-sectional views, depicting a percutaneous approach to gaining access to a subcapsular location.

FIGS. 10C-Z are cross-sectional, side and end views, depicting various approaches to controlling or engaging a capsule

FIGS. 10AA-AF are cross-sectional, side and end views, depicting various approaches to controlling or engaging a capsule.

FIGS. 11A-B are cross-sectional views, depicting a percutaneous approach to dissecting capsular tissue.

FIGS. 12A-B are cross-sectional views, depicting a percutaneous approach to accessing a subcapsular location and implantation.

FIGS. 13A-K are cross-sectional and partial cross-sectional views, depicting approaches to engaging or grasping a renal capsule or depicting spacers or grommets placed across a capsule.

FIGS. 14A-T are cross-sectional views, depicting various approaches to decapsulation.

FIGS. 15A-D are cross-sectional views, depicting an alternative percutaneous approach to device implantation.

FIGS. 16A-G are cross-sectional views, depicting functionality of renal interventional devices.

FIG. 17 is a cross-sectional view, depicting an approach to removing fluid from a kidney.

FIG. 18 is a cross-sectional view, depicting an apparatus for massaging or applying pressure to a kidney.

FIG. 19 is a cross-sectional view, depicting an alternative approach to removing fluids from a renal system.

FIGS. 20A-E are cross-sectional and perspective views, depicting further approaches to treating a kidney.

FIG. 21 is a cross-sectional view, depicting an approach to remove adipose tissue from the perirenal area.

### DETAILED DESCRIPTION OF THE DISCLOSURE

Before the present systems and methods are described, it is to be understood that this disclosure is not limited to particular embodiments described, as such may, of course, vary. It is also to be understood that the terminology used herein is for the purpose of describing particular embodiments only, and is not intended to be limiting, since the scope of the present disclosure will be limited only by the appended claims.

Where a range of values is provided, it is understood that each intervening value, to the tenth of the unit of the lower limit unless the context clearly dictates otherwise, between the upper and lower limits of that range is also specifically disclosed. Each smaller range between any stated value or intervening value in a stated range and any other stated or intervening value in that stated range is encompassed within the disclosure. The upper and lower limits of these smaller ranges may independently be included or excluded in the range, and each range where either, neither or both limits are included in the smaller ranges is also encompassed within the disclosure, subject to any specifically excluded limit in the stated range. Where the stated range includes one or both of the limits, ranges excluding either or both of those included limits are also included in the disclosure.

Unless defined otherwise, all technical and scientific terms used herein have the same meaning as commonly understood by one of ordinary skill in the art to which this disclosure belongs. Although any methods and materials similar or equivalent to those described herein can be used in the practice or testing of the present disclosure, the preferred methods and materials are now described.

It must be noted that as used herein and in the appended claims, the singular forms “a”, “an”, and “the” include plural referents unless the context clearly dictates otherwise. Thus, for example, reference to “the system” includes reference to one or more systems and equivalents thereof known to those skilled in the art, and so forth.

As disclosed herein, a renal treatment can involve one or more of the arterial or venous systems, approaches involving the urethra and or ureters, and percutaneous approaches. Moreover, each of the disclosed apparatus and methods can be adapted to accomplish desired treatments via arterial or venous systems, the urethra or ureters and/or percutaneously, or a combination thereof. Such treatment approaches can involve a guidewire, or can be adapted to be accomplished without a wire or guidewire. Further, each of the disclosed treatment approaches can involve employing vibration frequencies to enhance an ability to mechanically dissect a capsule from parenchyma.

Further, for all access, manipulation and/or piercing of renal tissue, it may be desirable to target the relatively avascular region of the kidney known as Brodel's line. Targeting this area minimizes the risk of bleeding. Accessing this area for procedural steps that require the most manipulation or highest risk of imparting some injury to the parenchyma will minimize the risk of bleeding. Conducting interventions in this location and then venturing outward along the kidney as required can allow those actions to be less traumatic. Further, all treatments can include addressing regrowth of tissue encapsulating the kidney.

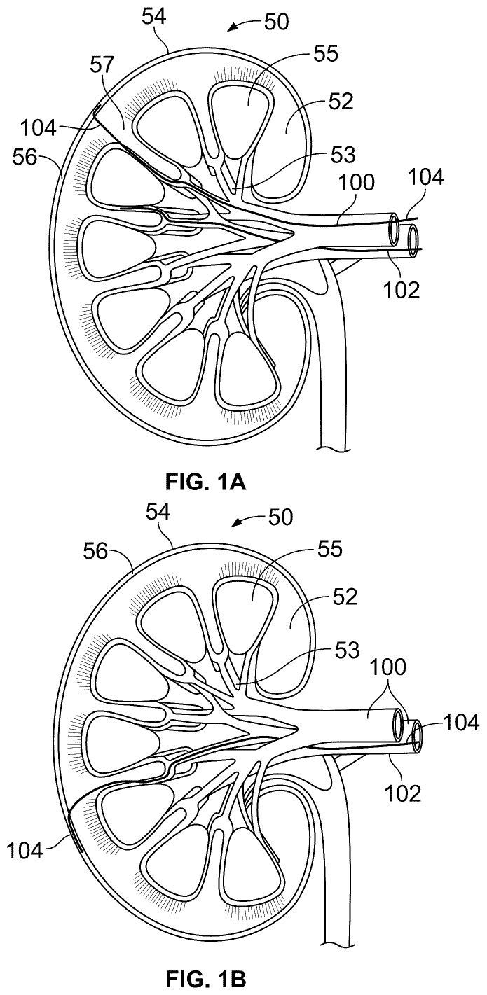

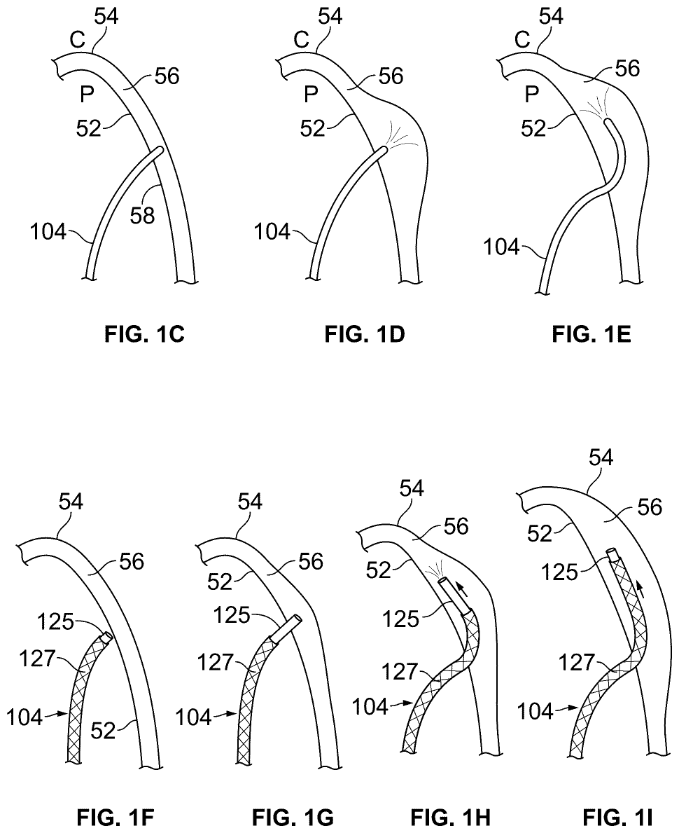

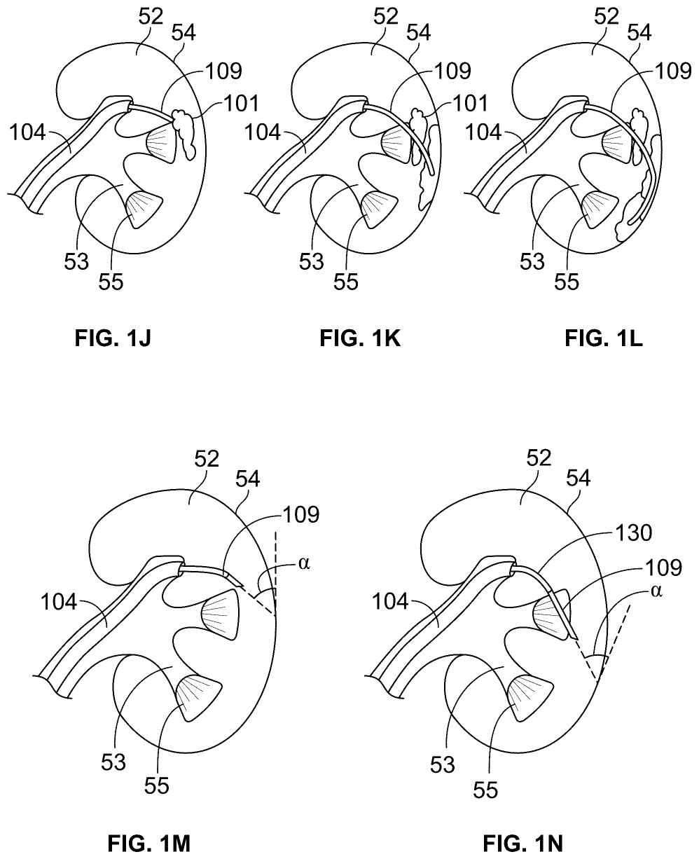

As shown in FIGS. 1A-B, a percutaneous vascular approach to a kidney 50 and its anatomy including the parenchyma 52, calyx 53, capsule 54, renal pyramid 55, subcapsular boundary or location 56 and renal columns 57, can be taken in a renal capsule access and/or pressure reduction treatment procedure. Through one or more of a femoral, brachial, radial or other vasculature of the leg, arm or neck site, access of a renal vein 100 or renal artery 102 is obtained such as by advancing an interventional device 104 therethrough. Access to the renal vein 100 or artery 102 can also be achieved via a trans-arterial or trans-venous approach by employing a trans-vascular needle or microcatheter advancement approach that facilitates jumping from one vascular site to another.

In one or more of the disclosed treatment approaches, a subcapsular location can also be accessed by using a percutaneous or laparoscopic procedure in which the subcapsular location is accessed from an extracapsular approach. An extracapsular approach can be from one or more of anterior, posterior or lateral approaches. Piercing into the subcapsular location can be achieved using a veres needle type or safety trocar type instrument that pierces the capsule without significantly or not at all engaging the parenchyma to avoid bleeding. An alternative is to use specially designed graspers to grasp an outer surface of the capsule and pull it away from the parenchyma surface slightly to allow penetration with a needle or a tip of specially designed scissors to create an opening in the capsule such as a hole or a slice along part or substantially all of a length of the kidney.

Turning to FIGS. 1C-E, access to the capsule 54 and subcapsular location 56 can be accomplished without use of a guidewire. In a first step, a treatment catheter or other interventional device 104 is advanced via vasculature or renal system anatomy to be positioned at the subcapsular location 56. Here, the treatment device 104 can be employed to perform diagnostic and/or therapeutic procedures. Alternatively, as described more below, the capsule 54 can be pierced from outside the capsule 54 using a percutaneous approach, and once positioned at the subcapsular location 56, the treatment device 104 can be used to perform diagnostic and/or therapeutic procedures.

As shown in FIGS. 1C-E, the treatment device 104 is advanced within the renal cortex or otherwise close to the parenchymal surface 58, and placed at the subcapsular boundary 56. The treatment device 104 can be configured to deliver pressurized fluid or other agents at the subcapsular location 56 to assist in piercing tissue and creating fluid dissection of the capsule 54 away from the parenchyma 52. In one particular or alternative aspect, the fluid employed in the dissection can be sucked or otherwise removed from the location, thereby leaving a space for kidney anatomy to expand into.

In one particular embodiment, the treatment device 104 can include microfluidic channels that deliver a liquid or gas to separate fibrous capsule layers, allowing controlled access without a physical dissection or puncture. In an alternative approach, a hydrogel or like material that will expand or swell can be employed to separate tissues and provide access to the capsule. In each of the disclosed embodiment, the treatment device 104 can alternatively or additionally be coated with a hydrogel to facilitate easy navigation within anatomy and thereby reduce a need for a guidewire or other platform for advancing the device within tissues. Moreover, in one or more embodiments, the tip of the device 104 can be constructed (tapered, beveled, angled or include thin-walled edges) to facilitate travel through the parenchyma or other structure.

The treatment device 104 can be configured to additionally or alternatively employ other means to dissect a space such as utilizing its own distal tip for blunt dissection or facilitate the advancement of a wire loop or other structures disclosed below. In such approaches, the treatment device is advanced and manipulated at the subcapsular boundary 56 to dissect tissue as desired to facilitate procedures for relieving renal pressures.

Accordingly, as with each disclosed embodiment, the treatment device 104 can be steerable such as including steering wires (not shown) incorporated into its structure, and further, can be configured to be flexible and torqueable as necessary to accomplish a desired procedure. Further, for each of the disclosed devices or approaches, adaptations or manipulations can be made so that the cutting of target tissue can be made in one or more of an antegrade or retrograde direction. Accordingly, where a device is described as being used in an antegrade direction, for example, it is to be understood that the device can be altered or manipulated to be used in a retrograde direction as well. Also, a device that is described as being used in a retrograde direction, it too can be adapted or manipulated to be used in an antegrade direction.

In one specific aspect, a treatment device can be a deflectable ureteroscope or a deflectable or pre-shaped catheter to be positioned via fluoroscopy or ultrasonography. Additionally, angulation and/or penetration can be achieved by nesting devices within a working channel of the treatment device. Further, a needle, straight or pre-shaped, could be first deployed and a microcatheter or implant deployed from therewithin.

With reference to FIGS. 1F-I, the treatment device 104 can include an inner catheter 125 coaxially arranged within an outer catheter 127. The inner catheter 125 is configured to deliver fluid from a distal end thereof for the purpose of creating a zone of dissection at the subcapsular boundary 56. The outer catheter 127 provides necessary columnar strength.

An iterative motion of the inner catheter 125 and the outer catheter 127 can be employed in an alternating fashion to provide support as the catheters are advanced within renal anatomy. Thus, the inner catheter 125 is advanced a given distance and used to dissect tissue and then the outer catheter 127 is advanced over the inner catheter 125 so that their terminal ends are arranged close to each other. The two catheters can then be advanced together to another location and the inner catheter 125 can again be advanced and used to dissect more tissue. Fluid ejected from the inner catheter 125 can be continuous or non-continuous along with advancement or other manipulation of the treatment device 104 for the purpose of dissecting or separating tissues.

With reference now to FIGS. 1J-L, there is shown one approach to treatment involving a steerable or shaped treatment device 104 that is advanced within renal tissue. The device 104 can be placed into a calyx 53 and a microcatheter 109 is advanced beyond the calyx 53 and into the parenchyma 52 (See FIG. 1J). The microcatheter 109 can have a radiopaque tip and embody an angled distal portion to aid advancement. An injecting contrast agent or gas can then be employed to create a dissection 101 within the kidney. The microcatheter 109 can then be advanced within the dissection space.

As the microcatheter 109 approaches the capsule 54, a dissection plane 101 is established via microcatheter injection (FIG. 1K). The fibrous nature of the capsule 54 and the preferential resistance of the dissection promote the deflection of the microcatheter off of the capsule 54 and along an internal plane. As shown in FIG. 1L, the microcatheter 109 can be advanced as far as desired within parenchyma 52. Thus, spaces are created for further interventional steps to achieve decreasing renal pressures.

An angle of approach to targeted tissue can be important in assuring maintaining a microcatheter within targeted renal tissues. An angle α between the microcatheter 109 and the capsule 54 of less than 90 degrees can be important in avoiding creating unwanted punctures in the capsule 54. To achieve desired angles, the treatment device 104 can be shaped or deflectable to direct the microcatheter to a desired angle relative to the capsule 54 or other anatomy (FIG. 1M). Alternatively, the microcatheter 109 itself can be deflectable to achieve the desired angles (FIG. 1N). Such arrangements can be incorporated into any of the disclosed apparatus to facilitate placement and navigation, whether the apparatus are advanced through arterial or venous system, lymphatic vessels, renal anatomy, or from an exterior of the kidney through a puncture of the capsule 54.

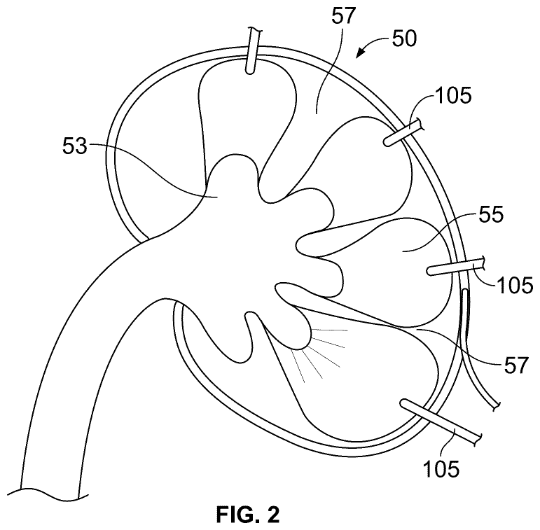

In one or more embodiments, multiple points of access or drainage apparatus are employed to reduce renal pressure. With reference to FIG. 2, the target renal anatomy can present a connected and integrally related interstitial pressure environment or can present multiple compartmentalized interstitial pressure environments. Multiple points of access and/or drainage apparatus 105 can be used to address elevated renal pressures where either connected and integrally related interstitial pressure environments or multiple compartmentalized environments are presented.

Where there is a compartmentalization of interstitial pressure environments, each compartmentalized section of the target renal anatomy can be treated individually or as a group or sub-groups with complementary methods and devices to relieve renal pressures with any of the disclosed interventional devices or methods. In one embodiment, an interventional procedure can involve directly treating or relieving the pressure of one or more parenchyma of a kidney or additionally or alternatively, with internal columns 57 or the areas between one or more calyx 53 or the calyxes 53 themselves (See FIG. 2).

In one or more aspects, a treatment procedure can be minimally invasive and thus no general anesthesia and/or open surgery is required. Here, only local anesthetics can be required. Other approaches can involve one or more of general anesthesia or surgery. In still yet other approaches, excess fat can be removed from within or around a target interventional site to reduce renal pressures or provide more space for renal fluids. Also, energy based interventional devices can employ energy to reduce the overall size of renal tissues to thereby reduce renal pressures.

A catheter approach can be employed in a treatment procedure. Also, multiple locations and actions and combinations thereof can be used to accomplish desired renal capsule access and pressure reduction. It can be advantageous to work in the venous system where there are lower pressures and/or less chance of hemorrhage. An arterial approach can also be employed as a treatment procedure dictates.

In one or more approaches, the renal artery 102 (or more distal arterial segment such as interlobular or arcuate) can be occluded during a treatment procedure to minimize hemorrhage. Such occluding can be timed based on a particular procedure and a need to most effectively minimize bleeding. When working on an arterial side, a renal artery catheter or a sheath through which devices can be deployed, can be used. In one embodiment, a sheath can include a balloon structure that is expanded to seal a vessel as desired, but allows catheters, wires or other instruments to pass to an interventional site. In this regard, the occlusion provided can be as distal as possible (such as interlobular, arcuate, afferent or efferent artery or vein) and to be adjacent the interventional site and in a common renal column.

Further, where an approach to the interventional site is arterial or both, there can be provided a working catheter with a balloon and subsequent wiring and devices can be passed through the catheter and be advanced as far as possible to occlude while directed treatment is accomplished through a distal or terminal end of the catheter. Alternatively, an occlusion can be created on a side or next to the working devices (See also FIG. 1A). When working through the venous side and in a same renal column that has an arterial supply, the occlusion catheter can be advanced into the column and occlude as desired.

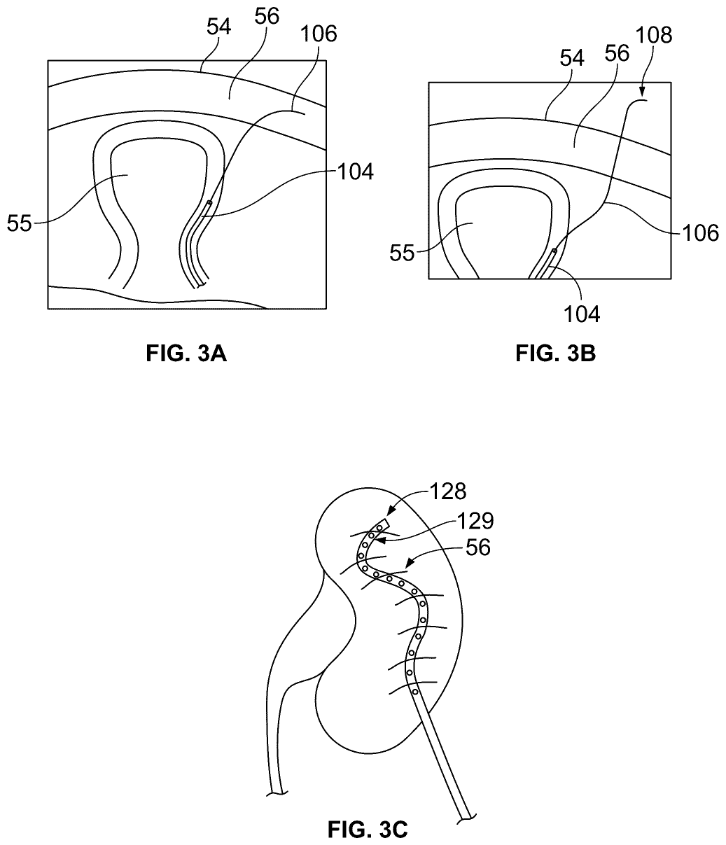

In one approach (FIGS. 3A-B), the treatment device 104 is advanced and then extra-vascularly or trans-vascularly positioned to access the subcapsular location 56. In one or more approaches, a wire or microcatheter (polyamide or other member structure) is then advanced to pierce a parenchyma 52 and to access a subcapsular location 56. In one or more further aspects the catheter can be passed into the dissected subcapsular space.

Once the subcapsular space is accessed, various different devices can be employed to modify the capsule 54 to accomplish one or more of decapsulation, renal release or pressure reduction and/or drainage into the renal, perirenal or peritoneal cavity. It is to be noted that each of the disclosed devices that are intended to ride on surface of parenchyma can incorporate smooth surfaces or other protective elements so as to minimize damage to the parenchyma as the device travels along the parenchyma, dissects the space, engages with the capsule.

In one particular approach (See FIG. 3C), subcapsular drainage of fluid can be facilitated by placing a drainage tube 128 within the dissected subcapsular space. The drainage tube 128 includes vented openings 129 configured to collect fluid. The drainage tube 128 can be placed into position over a previously placed guidewire or without the need of a guidewire. If a guidewire is used, after placement of the drainage tube 128, the guidewire can be removed and the tube 128 can be trimmed leaving its terminal end placed as desired. The drainage tube can thus be placed so that it provides a drainage path from the kidney to a collecting bag or to within retroperitoneal or abdominal spaces. Further, it may also be desirable to apply some degree of continuous, variable or intermittent suction to facilitate drainage.

In one particular aspect, a ballast system can be created such that the implanted tube or other interventional device is connected to a pressure-controlled valve that allows pressure to grow and be pushed outside of the kidney and into a balloon or other storage membrane that allows for better internal pressure regulation. A closed loop feedback system can additionally or alternatively be provided to incorporate a wirelessly controlled valve or shunt mechanism that can use artificial intelligence or at least a target pressure to change adaptively to pressure variations in the kidney, and to release pressure as needed during an interventional procedure.

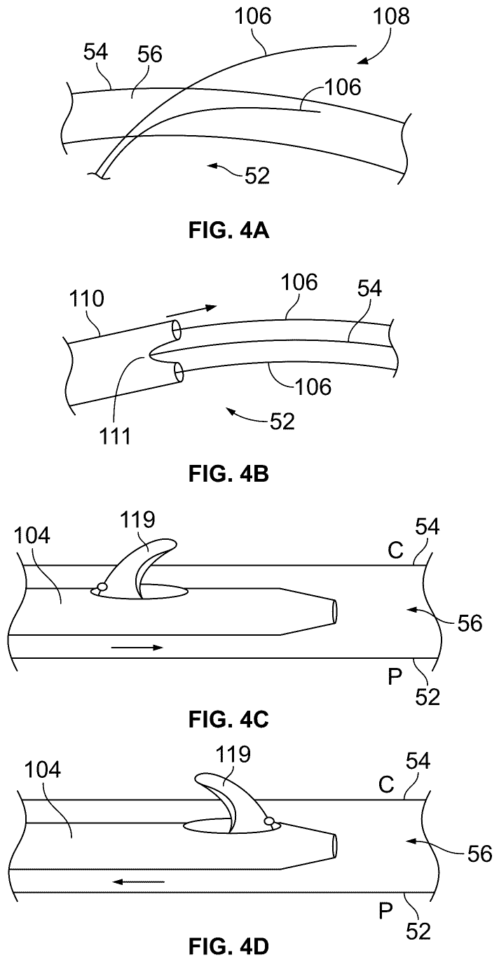

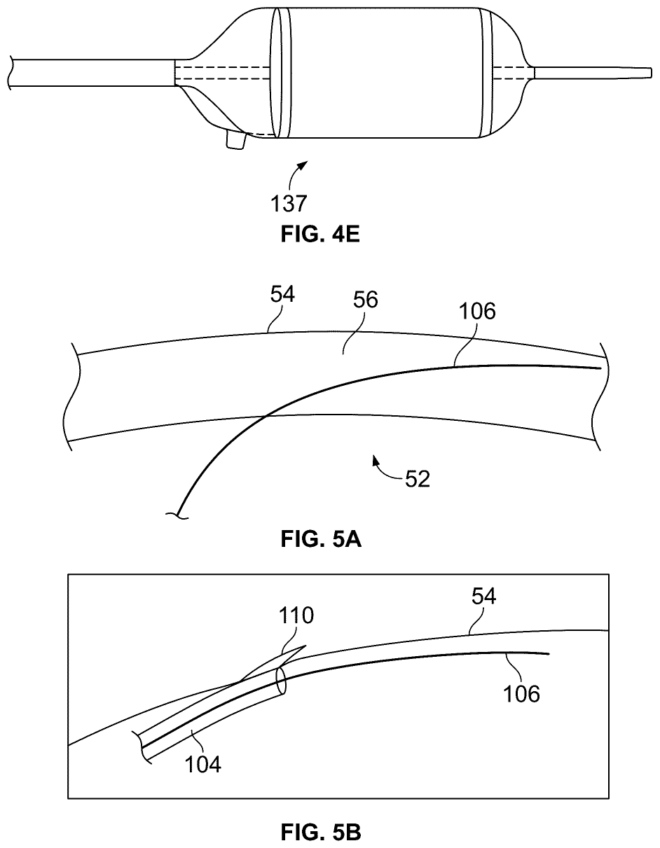

With reference to FIGS. 4A-B, in various approaches, there are provided methods and devices to accomplish linear capsular release. In one or more aspects, a wire or other member 106 is passed through the capsule 54 such as by making a hole in the capsule 54 and passing the wire or other member into a perirenal space 108. At least a portion of a second wire or member 106 is advanced and maintained in position in the dissected subcapsular space. In various approaches, the wire or member 106 is advanced as far along a desired path for release as desired. As shown in FIG. 4A, a first wire 106 is positioned within a dissected subcapsular space and a second wire 106 is advanced to within a perirenal space 108.

Once so positioned, the first and second wires 106 provide a platform for the advancement of an interventional device 110. The interventional device 110 can further include one or more cutting surfaces or a blade 111 configured to engage and cut renal tissue. In each of the disclosed embodiments, the dissecting or cutting structure can embody a mechanical structure and/or an energy-based mechanism (radiofrequency, heat, ultrasonics, microwaves, pneumatics, or lasers etc.) configured to cut tissue or create a puncture site. In one approach, the interventional device 110 is advanced along the first and second wires 106 with the cutting surface 111 positioned to cut the capsule 54. When the interventional device 110 has cut the capsule 54 as desired, it can be re-positioned to cut further renal tissue or can be removed from the interventional site.

In various aspects, capsular release can be accomplished by pushing and/or pulling cutting structures to make an incision in a capsule 54. The treatment device 104 can define steerable structure and include a leading edge that either rides on a wire or ejects fluid for forward dissection or blunt dissection. The device 104 can embody a fixed or deployable cutting edge that is one or more of mechanical, sharp or includes energy (RF, vibration, heat etc.) that can cut capsular or other tissue. The device 104 can be otherwise atraumatic to surrounding tissue except for the tissue engaged by the cutting structure.

In one or more embodiments, an alternative approach is to employ an IT knife (not shown) that embodies a monopolar electrode with an insulated ceramic backing to protect kidney anatomy. The knife is inserted at the interventional site (either from within or from outside the capsule) and the backing is used to create a controlled dissection of targeted tissue. An alternative is to utilize bipolar energy to create a controlled entrance and selectively cut through tissue. Bipolar energy can be created by using two electrodes that are placed on a surface of target tissue and advanced or retracted to create a dissection for example, while being insulated on a back of the device to protect other tissues from engagement with dissecting or cutting energy.

Such cutting structure can be deployable from within the treatment device 104 or may be oriented, extended or configured to a side of the device 104, and then rotated to reveal the cutting structure when needed. Advancement of the device 104 so configured accomplishes cutting the capsule 54 and releasing the parenchyma 52. The device 104 can be further configured to deliver a coagulating agent on the parenchyma 52 along and next to the path of travel of the device 104, for example when the device 104 is removed. A leading component of the treatment device 104 is sufficiently long and ahead of the cutting structure so that the device 104 stays between the capsule 54 and the parenchyma 52. Further, the device 104 embodies sufficient stiffness to not flex back on itself and flip out from under the capsule 54, and is atraumatic in shape and material to not increase the risk of bleeding.

In one particular embodiment (See FIGS. 4C-D), an interventional device includes a sharpened fin or blade that is configured to be dragged or advanced along capsular tissue to accomplish decapsulation. The sharpened fin or other cutting structure can be movable or configured to be retracted when not in use or engaged. In one or more embodiments, the energy or mechanical cutting devices can include or be associated with structure to retract, cover or otherwise shield the cutting element from inadvertent damage to non-targeted tissue. This can be in addition to any features that help guide the device and protect/shield damage to non-targeted tissue during a cutting or other action.

In one specific approach, the treatment device 104 includes a cutting fin 119 that can be employed to release the capsule 54 along a dissection tract about the kidney 50. Once the device 104 is positioned in the desired treatment location, the cutting fin 119 can be used to cut tissue via a pushing motion (See FIG. 4C). The cutting fin 119 can be fixed or can be deployable from within the treatment device 104. When fixed, the cutting fin 119 can be positioned to the side such that it does not interact with the capsule 54 or parenchyma 52 as the device 104 is advanced. When needed, the device 104 can be rotated or deployed to present the cutting fin 119 in a manner to cut tissue or engage the capsule 54.

Once deployed or positioned for engagement of tissue, the fin 119 can be used to pierce the capsule 54 mechanically by via energy or vibration. As the treatment device 104 is manipulated, the fin 119 cuts the capsule thereby releasing the parenchyma. Notably, the treatment device 104 can further include structure that rotates the fin 119 so that its cutting edge is also presented at various alternative angles for cutting tissue (FIG. 4D). Alternatively, the fin 119 can be fixed in a rearward-facing orientation for cutting tissue.

In one embodiment (See FIG. 4E), an ablation device can be employed to weaken or dissect a capsule or can be otherwise used facilitate drainage of fluids. The ablation device can be embodied in an ablation balloon 137 which is positionable within a subcapsular location, allowing precise placement against an inside wall of the capsule. The balloon 137 is activated to deliver energy such as thermal or cryoablation energy to the inner surface of the capsule, which operates to weaken or modify the tissue structure of the capsule without causing damage to outer layers of the kidney. The same may involve thermal damage or freezing of targeted tissue which leads to alternations in the collagen fibers and structural components of the capsule. Here, one goal is facilitating subsequent interventions such as drainage procedures, dissection or resection of the targeted renal tissues. By so weakening the capsule, for example, further specific therapeutic action can be aided without a need for more invasive surgical techniques. Such methods benefit from being repeatable, inducing controlled damage to targeted tissue, reduction in scar formation and allowing for outpatient procedure with quicker recovery.

The balloon structure 137 can also be configured to provide cryo-ablation without using energy. In one or more aspects, the balloon structure can be embedded with ablation or electro-cautery mechanisms. The balloon 137 can be inserted via a guidewire, expanded, and then energy channels configured on the balloon are configured to treat targeted tissue. The energy emitted is directed at the target tissue such as only from one side of the balloon 137. When activated, the energy causes tissue dehiscence and can, for example, cause the capsule to disintegrate over time and thereby reducing the ability of the capsule to apply pressure to other renal tissues.

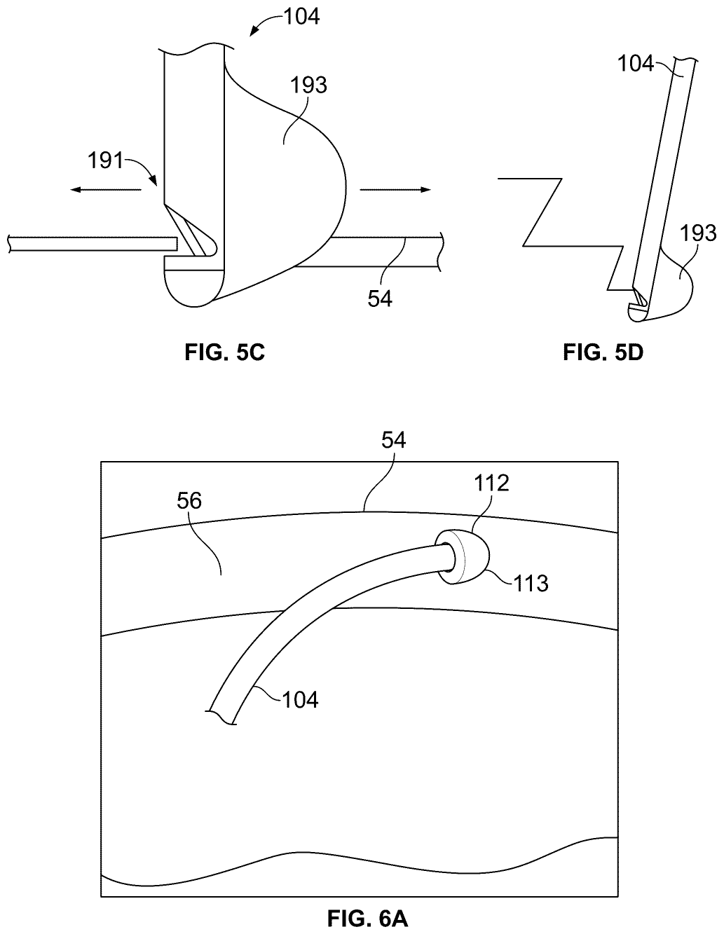

Next, with reference to FIGS. 5A-B, an interventional device 110 is passed over a wire or member 106 positioned as desired within renal tissue until it engages the hole made by the wire or member 106. Alternatively, the interventional device 110 is configured to create its own hole or path through the targeted renal tissue including the capsule 54. As shown in FIG. 5A, in one approach, the wire 106 is positioned across a parenchyma 52 with its distal end portion configured within a dissected subcapsular space.

The interventional device 110 can then be employed to operate as a releasing catheter and manipulated to zip or pass along and cut the capsule 54 along a path provided by the wire or member 106. Alternatively or additionally, the interventional device 110 can operate independently of the wire or member 106. The interventional device 110 can include mechanical cutting structure 111 or can employ energy such as radio-frequencies, heat, ultrasonics, or lasers or the like to accomplish desired cutting or re-forming of the capsule. The interventional device 110 and wire or member 106 can be utilized multiple times and in various directions to complete a treatment procedure. In one aspect, the wire or member 106 is redeployed to provide a path for the cutting structure, or the interventional device 110 with the cutting structure is deployed independently.

A tissue cutting motion can involve an advancement of a cutting implement over a wire or independently, or can be a retraction of the cutting implement over a wire or not. In one particular approach, an engagement of tissue or a purchase relative to tissue is gained or accomplished and then the cutting implement of an interventional device is advanced to the target tissue to be cut. Next, the cutting implement can be pulled proximally to cut or release targeted tissue while taking advantage of the purchase maintained relative to the targeted tissue.

It is to be noted that in one or more approaches or embodiments, both pulling and pushing release of a capsule can be from under the capsule or from outside the capsule. For example, a treatment device can hook into a capsule and be pulled from the outside along the capsule to affect a cut.

In one or more embodiments, the interventional device 110 can run over a subcapsular wire or an extracapsular wire 106. The device 110 embodies structure that is sufficiently torqueable to control as it is employed at a treatment site. The device 110 additionally or alternatively can embody a dual lumen structure 114 (FIG. 4B) configured to provide access to one or more or both of a subcapsular and extracapsular wire or member 106 to thus ensure that the cutting or capsule engaging structure 111 of the device 110 is oriented to engage the capsule 54 as desired and directed.

It is to be noted that the capsule 54 can be any structure that is associated with a kidney that partially or wholly contains fluid that might buildup within or about a kidney, or otherwise constrains kidney tissue from expanding. Thus, the capsule 54 referred to herein can be native capsule tissue or new capsular growth or other tissue response that partially or fully encapsulates the kidney.

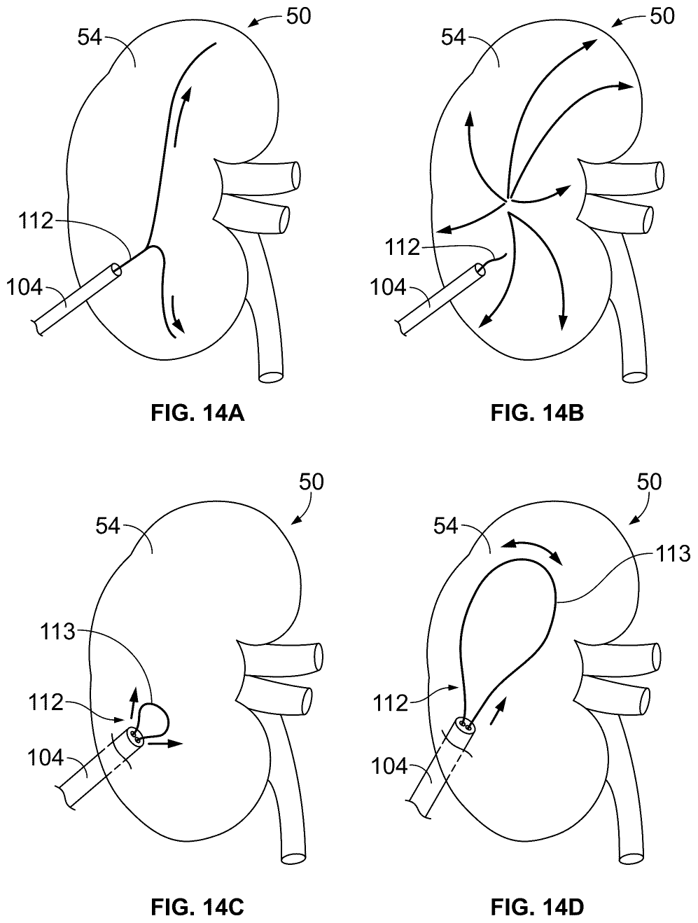

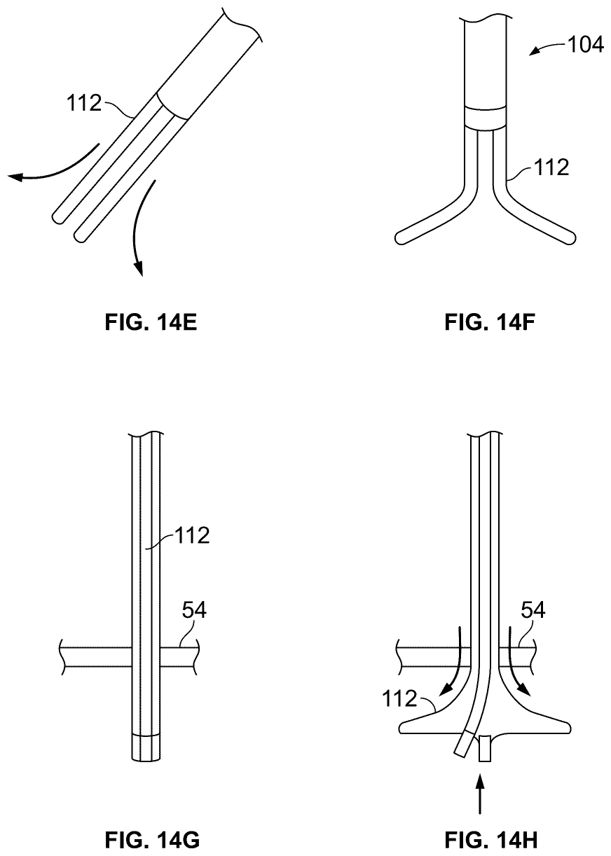

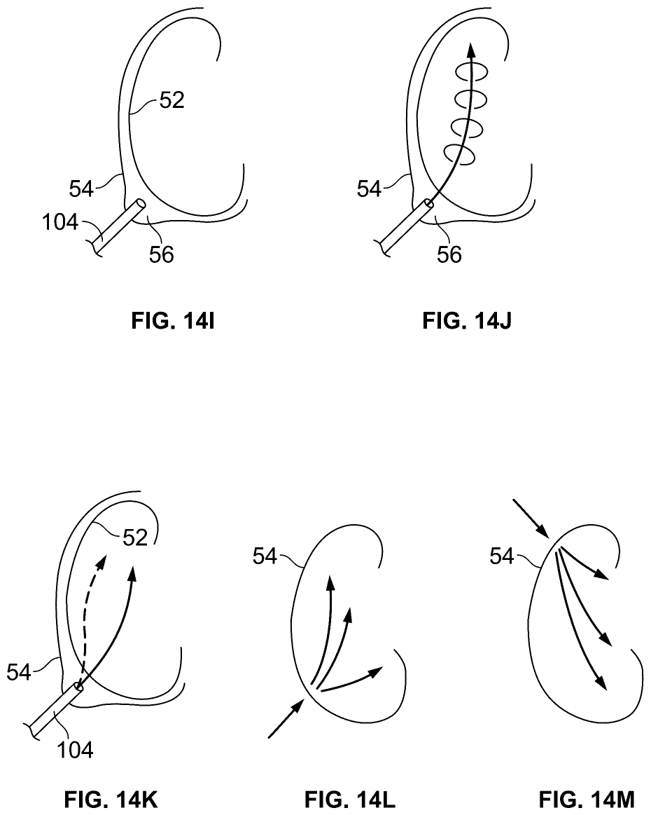

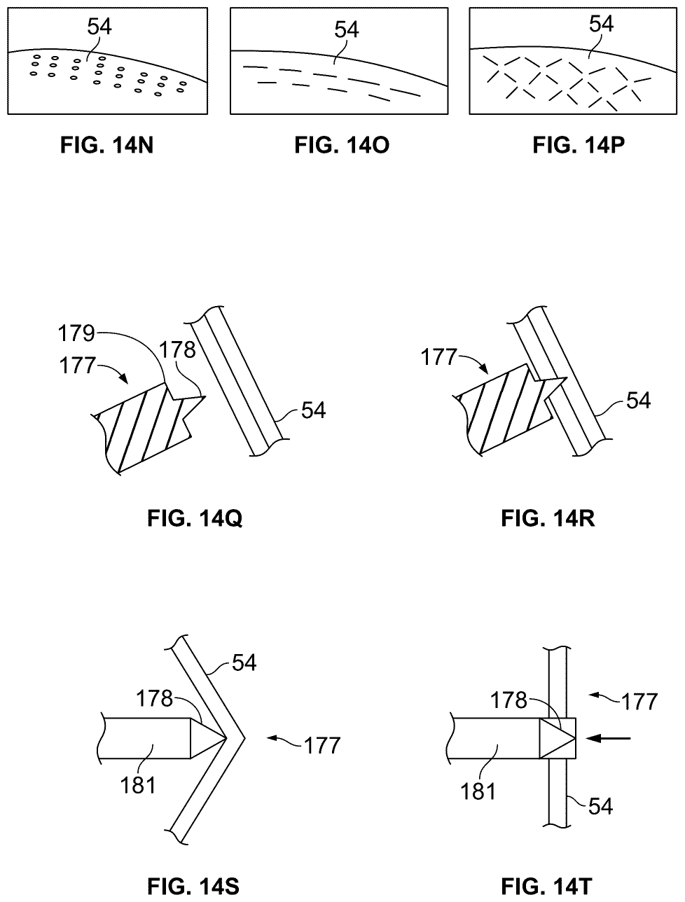

Further, in one or more embodiments, the interventional device 110 can be additionally or alternatively configured to include structure providing protection so that non-target tissue such as the parenchyma 52 is not cut or engaged. In this regard, the device 110 can be biased or include geometry that facilitates safety such as by incorporating safety structure in the head or along a body of the interventional device 110 (See also FIGS. 14Q-T). There can additionally or alternatively be orientation markers incorporated into the interventional device 110 to facilitate proper placement relative to a treatment site, or the device 110 can additionally or alternatively include sensors providing one or more of positioning, torquing, stress or engagement force information.

In one particular approach, a balloon assembly can be employed to engage, dissect, cut, reconfigure, release and/or otherwise treat a capsule for the purpose of reducing pressures or fluid build-up. The balloon assembly can include one or more of wires, blades or other tissue cutting or scoring elements. In one aspect, such structure is configured on a known side of the balloon of the balloon assembly. The balloon itself can assume a generally flat profile or a catheter where a distal end has an aspect ratio that is other than 1:1 so there is a clear wider side like a paddle structure. The balloon can also be configured to be directional, that is, the balloon presents an asymmetrical profile and/or include a hard or rigid side or backing so that when it is expanded, the balloon can better protect certain tissues such as internal organs, from potential pressures and/or resultant damage during dissection. Once advanced and inflated or deployed at a treatment site, the blades or wires or other cutting or scoring elements are preferentially positioned to face capsule anatomy and then manipulated to cut or score the capsule to either open or weaken the capsule for potential stretching or separation of the capsule under pressure of the balloon. In an alternative or additional aspect, the balloon assembly can embody heat or other energy producing elements to effect a desired change in the integrity, strength or other properties of the capsule.

In one embodiment, the balloon assembly defines a balloon dissector (not shown) configured to separate the renal capsule 54 from parenchyma 52 generally or where there are adhesions between the two structures. Such separation or dissection can be necessary when a capsule 54 grows back and repeat decapsulation is required, or where other disease mechanisms affect the capsule 54 or cause unwanted adhesions. In this regard, a guidewire can be advanced to a treatment site to provide a path for placing the balloon at the treatment site. As with each of the disclosed approaches to treatment, the treatment procedure can also be performed without the use of a guidewire. The balloon is then used to separate tissue. Here again, balloon can assume a flat profile or can embody a winged structure that unfurl when inflated.

Further, with reference to FIGS. 5C-D, a distal end portion of a treatment device 104 can include a cutting assembly 191 that is biased against target tissue by a balloon or other expandable member 193. Such an assembly can be used where there is limited access to a target capsule 54, for example. In various embodiments the balloon itself can define cutting structure or blades can be attached thereto. Moreover, as shown, the balloon 193 can be configured and used to drive the cutter 191. In one approach, backed against renal anatomy and pushing into the capsule 54, the inflated balloon 193 presses against the capsule 54 on one side and drives the cutter 191 into the capsule 54 on the other side to create a cutting path. As so arranged, a zigzag or other desired cutting path can be created with each inflation or partial inflation of the balloon 193.

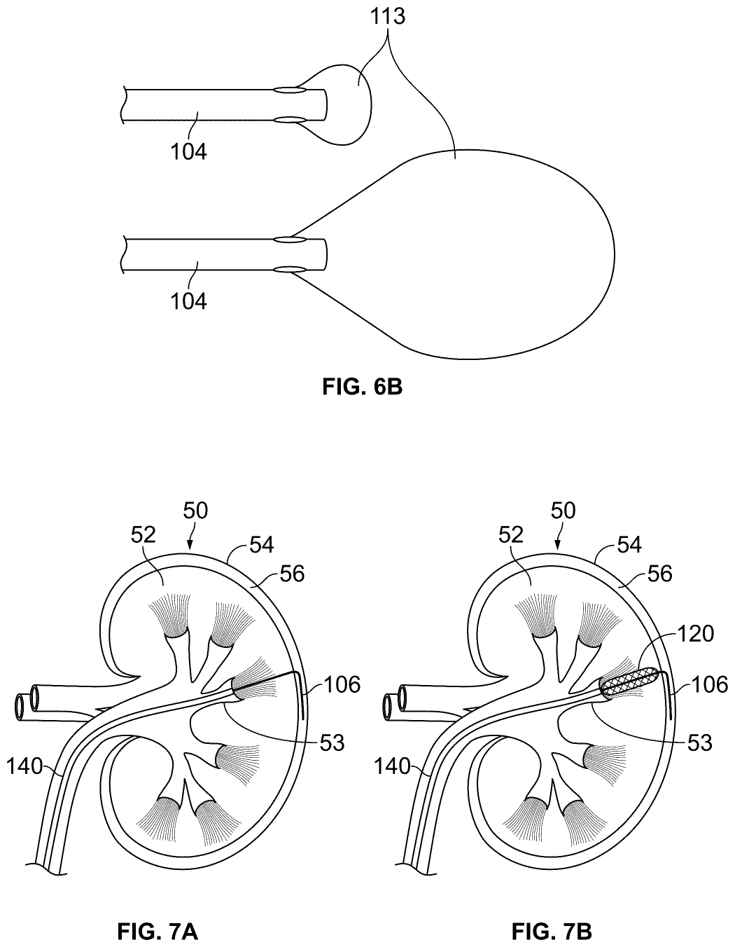

In another approach (See FIGS. 6A-B), an interventional device can embody a catheter 104 with a wire 112 that is manipulated to define a distal loop 113. This approach to an interventional device or catheter 104 can be additionally or alternatively used in a treatment procedure to reduce pressures or fluid build-up in renal anatomy. The loop 113 can be deployed at the treatment site and advanced and manipulated to enhance creating a dissection plane between parenchyma 52 and capsule 54 tissue structures. In one approach, the catheter 104 and/or loop 113 can be formed from nitinol.

As shown in FIG. 6B, the wire 112 is advanced to make the loop 113 larger to thus present a profile for separating a capsule 54 from a parenchyma 52. Such structure can additionally or alternatively be used to cut the capsule 54 or other renal tissue. A mechanism that generates vibratory or other energy can further be incorporated into the interventional device 104 to aid the device in separating or cutting tissues. Moreover, various coatings can be applied to the loop structure 113 to assist in separating or cutting tissues.

In a particular approach, the interventional device can be steered in the subcapsular space to assist in dissecting the capsule from the parenchyma as well as assist in directing any wire or other device. In this way, the device can sweep side to side as necessary with enough force to dissect the capsule. Moreover, the operator can then align a catheter along any desired trajectory for dissections. Here also, the device can employ energy, vibration, lubricious coatings or other to enhance dissection, tracking, advancement or the like.

In a further additional or alternative aspect, a wire or various other structures can be passed or advanced and manipulated at a treatment site to pierce the capsule. Energy modalities can be additionally used to facilitate maintaining holes created through the piercing action. This can be repeated multiple times to pierce, slice or fenestrate the capsule for desired reducing of pressures or fluid build-up.

In a further additional or alternative aspect, a morcellator or various other structures can be passed or advanced and manipulated at a treatment site to remove a portion of the parenchyma along the inner wall of the capsule to create a small cavity or space within the capsule to allow a little expansion of the remaining kidney tissues in order to reduce the pressure on those tissues within the capsule. Energy modalities can be additionally used to facilitate the removal of and cauterization of parenchymal tissue. This can be repeated multiple times for desired reducing of pressures or fluid build-up.

Once the capsule is vented or pierced or treated as desired, excess fluids can drain into one or more of a perirenal 108 or a peritoneal cavity so that the fluids can be re-absorbed by the body. Such fluids can also be withdrawn from the body or re-inserted into vasculature using natural pressure gradients or a pump. In one approach, a three-way valve connected to a pump is provided to facilitate pumping of fluids to various places. The pump can be configured to cooperate with the patient's diaphragm such that as the diaphragm moves, the pump operates to withdraw fluids from the interventional site.

In various approaches, to accomplish capsule venting or perforating, a subcapsular wire or catheter or other interventional devices can involve mechanical approaches to engaging the capsule. Angled or deflectable devices or catheters can be employed and wire or other capsule engaging structures are driven out to cut or pierce a capsule. As stated, the capsule engaging structures can additionally or alternatively include energy producing modalities to add in affecting the capsule as desired.

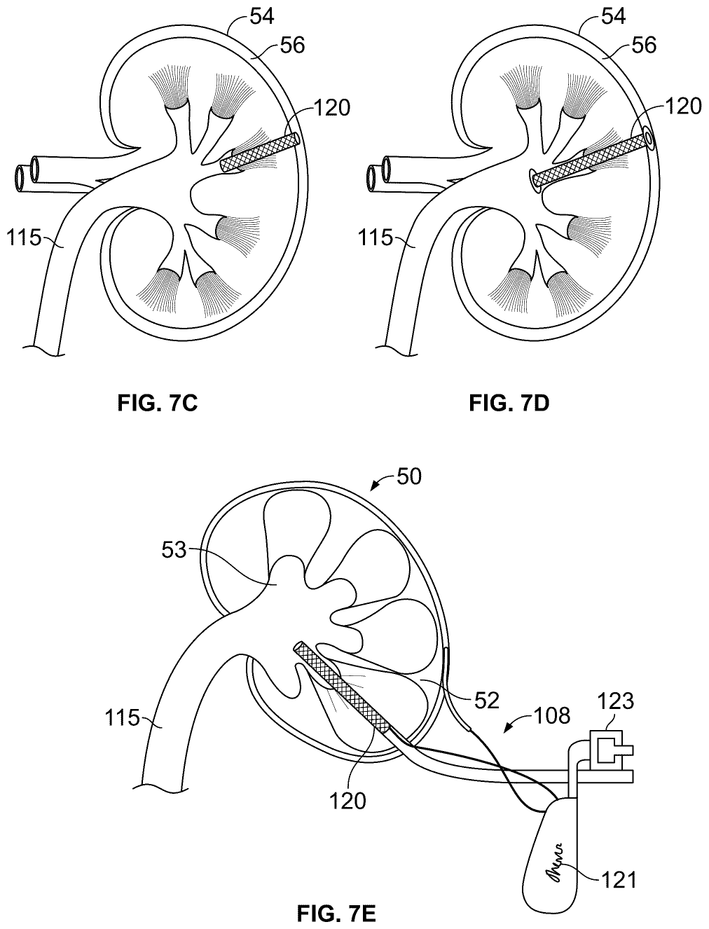

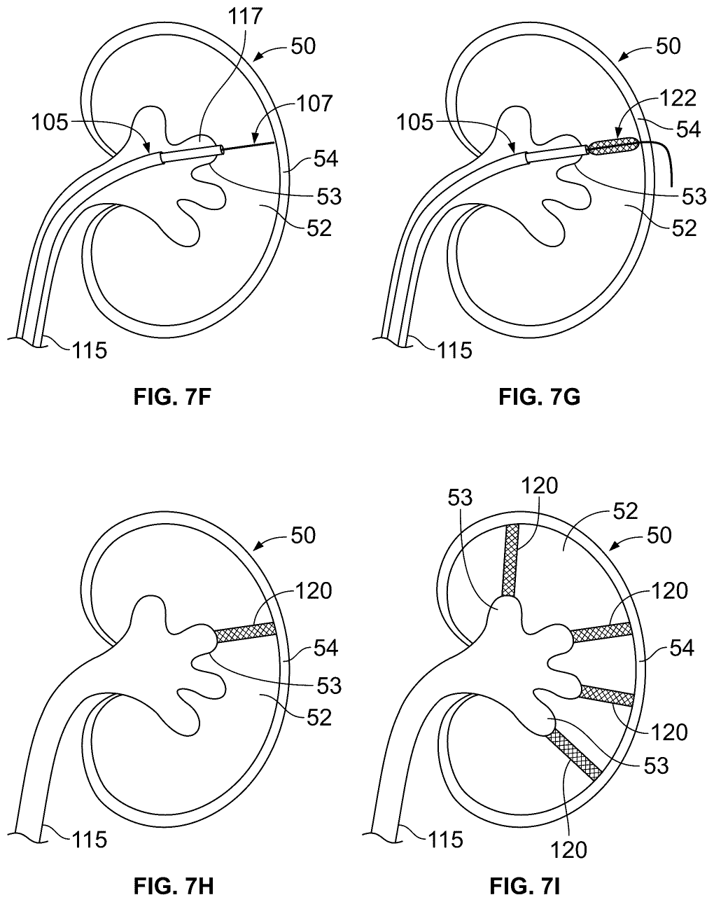

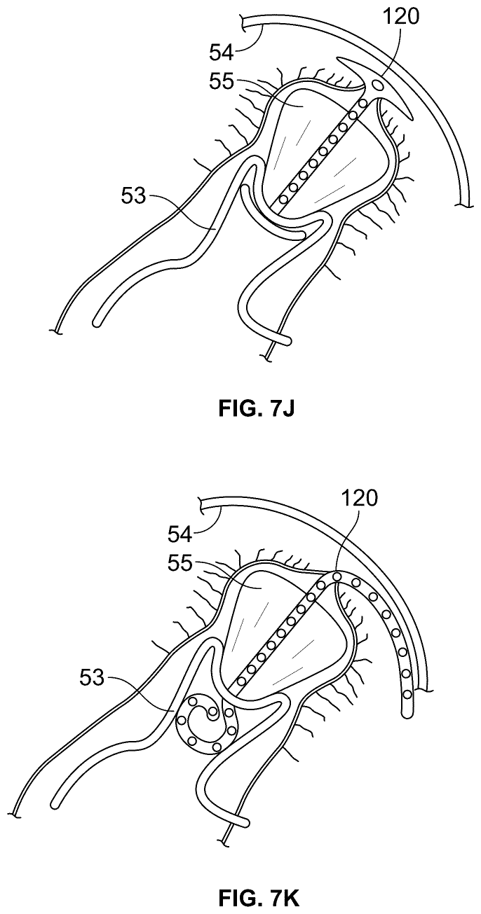

Alternatively or additionally, a ureteral approach such as through a ureter 115 can be taken in a renal capsule access and/or pressure reduction treatment procedure (See FIGS. 7A-E and 8A-D). Each of the herein described devices or assemblies can be used or modified in an interventional procedure while taking the ureteral approach. Thus, access to the target anatomy can be via the bladder and/or ureter to the renal pelvis and beyond. In one embodiment, a catheter-based approach can be employed where a typical ureteral sheath can have diameters ranging up to and beyond 12-15 French outer diameter and 10-13 French inner diameters.

In this approach, one objective is to create a shunt from the renal calyx 53 into a subcapsular location 56. The pressure of kidney calyces and urinary tract are reliably low unless the patient suffers from chronic urinary retention or other obstructive pathologies, which are pathologies generally independent of congestive heart failure. Thus, a pressure differential will drive interstitial fluid along an implanted shunt to the urine drainage space. Notably, by remaining subcapsular, the risk of retroperitoneal bleeding is minimized.

To combat negative effects of elevated intrarenal interstitial pressure, both kidneys can be addressed as they will likely be similarly affected by pathologic pressures created by heart failure. Bilateral access to the kidneys is routinely accomplished through ureteroscopy, and a bilateral procedure, or even staged lateral procedures, may be more feasible and less complicated than if conducted from arterial, venous or lymphatic vascular access.

The drainage system of the renal pelvis offers more space than the vascular supplies to place, steer or implant devices and to control device trajectories. Additionally, much larger devices may be utilized to deploy smaller devices from within. Targeting shunt placement through the calyx and along Brodel's Line may minimize intrarenal hemorrhage as well as clot occlusion of the shunt, and may minimize interference with the renal architecture. If the shunt needs to be examined, repaired, replaced, or removed, ureteroscopic access is readily conducted.

Since normal inter-renal interstitial pressure is positive but very close to atmospheric pressure (estimated to be approximately 2 mmHg), if heart failure arterial and central venous pressures are later managed closer to normal and less fluid enters the intrarenal interstitium, flow through a shunt will naturally reduce or cease due to the very low pressure differential between interstitium and a drainage system. While encrustation and stone formation is a concern in the collecting ducts of the kidney, because the shunt will be carrying interstitial fluid, rather than urea concentrated urine, it may be less prone to encrust. In fact, this fluid may be protection against this process.

A shunt can be a long drainage tube that courses along the subcapsular space, or can be a short connection between calyx and a single location in this space. Shunts can be an implanted foreign material such as metal or plastic, or can be a surgically constructed channel, either through simple piercings that may heal over time, or denatured tissue channels created with means such as radiofrequency or lasers.

Alternatively or additionally, shunting may be established primarily or even exclusively within the renal medulla. Perfusion of the medulla can be worse than that of the cortex in a congested kidney. Additionally, biomarkers can show preferential damage occurring to renal tubules over glomeruli. Also, anatomically the medulla has less lymphatic drainage available than cortical tissue. For an implant, or surgically created shunt, the shorter distance required of medullary shunting can be attractive with regard to simplicity, reliability, patency and removal.

In one approach, a working sheath 140 can be configured to be advanced through a ureter 115 and positioned with its terminal end near an entry point into the parenchyma 52 from the calyx 53. In one specific aspect, the working sheath can be a deflectable or pre-shaped uretoscope that is advanced into the kidney via the urinary tract, and where possible along the Line of Brodel to minimize vascular damage or bleeding. A wire or catheter 106 is then driven across the calyx 53 into the subcapsular location 56 (FIG. 7A). Notably, the interventional procedure can alternatively be conducted without the wire 106.

Under guidance such as fluoroscopy, the treatment device 104 can be used to inject a contrast agent or gas to create a subcapsular dissection plane. Alternatively, the space can be dilated if necessary using a balloon structure. Under fluoroscopy, the dissection plane can be seen along the tip of the treatment device 104. A shunt, stent or tube 120 may be inserted in the space created (FIG. 7B) by extending it out of the treatment device 104. Alternatively or in conjunction, energy (RF, cryogenic or other) can be utilized to create a channel to allow for any lymph or fluid to drain from the subcapsular space into the calyx 53 (FIG. 7C) from which the lymph or fluid can be voided with urine output. The energy created channel can seal and create a durable shunt 120 without a need for an implant, but an implant can be added when deemed necessary.

In an alternative or additional aspect, a shunt implant 120 can be used to maintain the path created between the calyx and subcapsular space (See FIG. 7D). The established path allows excess interstitial fluid to pass along the pressure gradient from interstitium to the collecting duct. This may come from the cortex, medulla or both, depending on the implant configuration. Side holes or porosity of structure allows for interstitial fluid to enter the shunt from intermediary zones of tissue. As stated, the shunt 120 can be formed of a polymer (i.e. silicone) or from metal (i.e. stainless steel, nitinol).

The shunt 120 can embody anchoring features at its ends or along its length to prevent migration such as flanges, barbs or hooks that sit on the parenchymal surface or other flange or channel, wedge or diameter changing structures that facilitate a robust engagement. In one particular approach, anchoring structure can be embodied in an expandable or self-deploying balloon (not shown). Such expansion of shunt structure can help with hemostasis. Moreover, the shunt or stent structure 120 or other contemplated implants, can be coated with an agent or can be drug eluting to further assist with hemostasis or other therapeutic goal such as preventing unwanted re-growth of tissues.

Additionally, as shown in 7E, the stent or shunt 120 can have a length or can be positioned so that it extends from within the calyx 53 through the parenchyma 52 and into the peri-renal space 108, or beyond and reach outside a patient's body. The shunt 120 can be connected so that collected fluid drains into the ureter and out the body. In this regard, fluid can be caused to go into the calyx 53, into a bladder (not shown) and then out of the patient's body, or fluid can be directed to go out to a perirenal space and then out of the patient's body. A one-way valve can additionally or alternatively be provided to maintain a unidirectional flow and steady state pressure environment in targeted tissue. Further, a drainage tube (not shown) can be configured, without violating a parenchyma, from a subcapsular space and into a collection system or reservoir outside or within the patient's body. Here, a path to outside the patient's body can be taken through the patient's ribs or back generally.

Alternatively or additionally, the shunt 120 can be connected to a collection bag or reservoir 121 configured within the perirenal space. A needle (not shown) can then be used to withdraw collected fluid from the collection bag 121 periodically via a subcutaneous located port 123.

In related approaches, a stent or shunt or other implant 120 can be placed in one or all of the anatomical structures of the kidney 50 to effectively reduce or relieve renal interstitial pressure as those areas which all drain into the same calyx 53 may not easily fluidly communicate with one or another tubular group region. In one particular aspect, the implant 120 can be customized for a pre-determined length measurable by various means such as via radiopaque markers configured on a wire introducer or a wire itself employed to deliver the implant 120, so that a proper stent length and delivery balloon may be selected. In another aspect, a marker of known length can be configured on an introducer assembly so that a local measurement can be approximated by measuring the marker on an image of the interventional site, and comparing it to a length of a delivery wire 106 as it traverses from a calyx 53 to a capsule 54.

Further, in one or more embodiments, the implant 120 can be configured to define separate links with discrete lengths so that a certain number (lengths) can be deployed and release at the time of a treatment procedure without a need to select just one pre-determined length. In this specific regard, the interventional treatment can involve delivering a series of independent implants 120 loaded in a delivery sheath over a balloon where a degree to which the sheath is withdrawn determines a number of implants 120 that are being deployed. Alternatively, a known or desired series of pre-loaded stents are configured to be so deployed.

With reference now to FIGS. 7F-G, there is shown an interventional system which can be used to deliver an implant or stent 120 configured to be completely contained within a parenchyma of a kidney 50. In a first step (FIG. 7F), a guide catheter 105 is placed through a ureter 115 or other anatomy to gain access to an interior of a kidney 50. As shown, the guide catheter 105 is placed so that its terminal end resides within a calyx 53. As so positioned the guide catheter 105 can be employed to introduce further interventional implements. In particular, a set of introductory and support catheters can be provided to allow the interventional system to be set with a relatively stiff guide-base to allow introduction of the interventional system into the parenchyma without it backing out. Accordingly, in a first step, a wire introducer 117 can be advanced through the guide catheter 105 and positioned beyond the calyx 53 and into a parenchymal space 52.

Next, a modified blunt tipped wire or a tight “J” tipped wire 107 is advanced through the guide catheter 105 and wire introducer 117, and configured and employed to guide delivery apparatus to stop when the capsular layer is reached. As shown in FIG. 7F, a terminal end of the wire 107 is positioned adjacent the capsule 54.

A broad-shouldered balloon catheter assembly 122 is then advanced over the wire 107 and configured within the parenchyma 52, where it can be dilated to create a desired space (FIG. 7G). The broad-shouldered design can provide dilation and delivery that minimizes a distance between a peak diameter section of the balloon and the terminal end of the balloon catheter assembly 122. The balloon catheter assembly 122 can be used to deliver an implant 120 or it can be exchanged with another balloon catheter assembly to deliver a stent.

Notably, the previously placed wire 107 guides the balloon assembly 122 to within the parenchyma 52 just short of the capsule 54. Further, the implant 120 can embody with wide enough interstices that facilitate the passage of fluid and which present a strong enough structure to maintain an expanded dimension. In an alternative approach, the implant 120 can be self-expanding thus eliminating a need for pre-or-post-balloon expansion and the balloon expansion, if necessary, can be only used for one or more of pre-or-post stent expansion. Also, the implant 120 may require a balloon expansion but the balloon can be separate and introduced into the implant 120 after the implant 120 is positioned in the tissue, or it can be pre-loaded on the balloon. Here also, a coating on the stent or other implant such as that used in coronary stenting, can be provided to prevent formation of an endoluminal coating over the now exposed parenchyma which would maintain the opening as a site for excess intersitital fluid drainage.

As shown in FIG. 7H, a shunt or stent 120 can thus be implanted entirely within the parenchyma 52. A distal terminal end of the stent 120 is configured short of the capsule 54, and a proximal terminal end of the stent 120 is configured to be aligned to an edge of a calyx 53, precisely terminating at a transition of the calyx 53 into the open space of the calyx 53. Moreover, as shown in FIG. 7I, multiple implants 120 can be so placed in a treatment procedure. In this way, renal interstitial pressures can be reduced or relieved as desired.

In one particular approach (See FIG. 7J), the implant 120 can be positioned so that it crosses and extends just beyond a renal pyramid 55. Potentially made of elastic biocompatible material such as silicone, the ends of the implant 120 can deploy from a treatment device. Since the calyx 53 is widest at the renal pyramid 55 border, it may be that an expandable end is not needed in the parenchyma end of the implant 120, since the calyx 53 end will be held within the anatomical structure itself. The calyx 53 end of the implant 120 can be porous or assume a low profile so that normal exudate continues to be allowed to enter the calyx 53.

Referring to FIGS. 7K, an implant or shunt structure 120 can embody a drainage catheter that has a pre-shaped proximal end, such as a pigtail, configurable within a calyx 53. The implant 120 can be configured to cross the renal pyramid 55 with its distal end portion positioned along the renal cortex or capsule 54. Here also, the implant 120 can include a plurality of holes or define porous structure adapted for drainage along a portion of its length.

As shown in FIGS. 7L-M, a shunt or implant 120 can be configured along a Brodel's line (BL). In this regard, a single calyx 53 can be involved in shunting and the implant 120 can be placed subcapsularly along a Brodel's line, again to minimize trauma and bleeding.

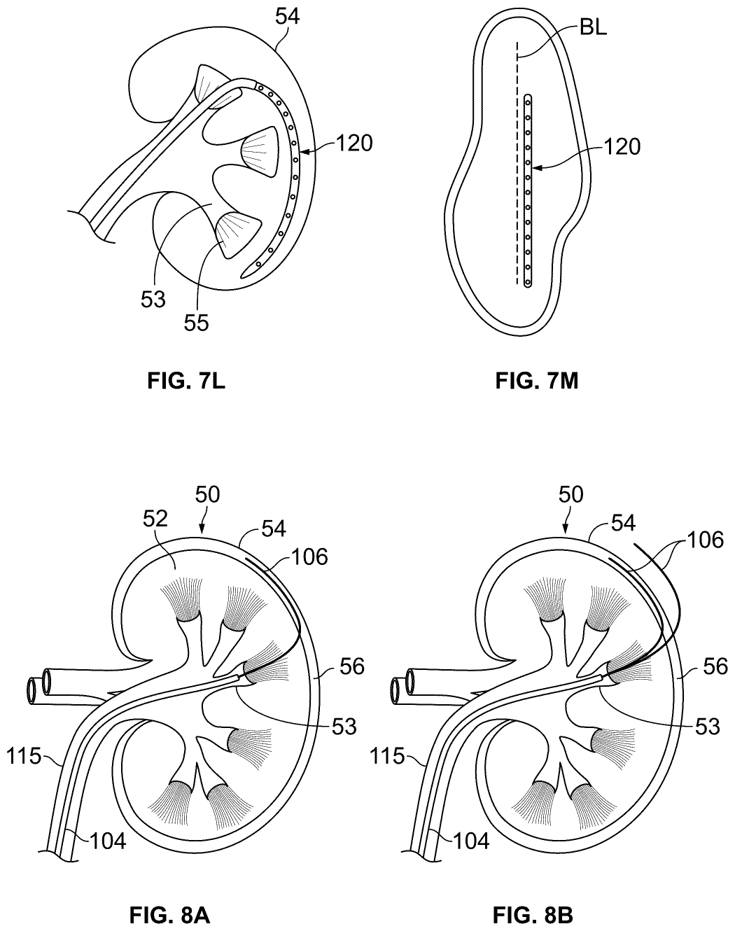

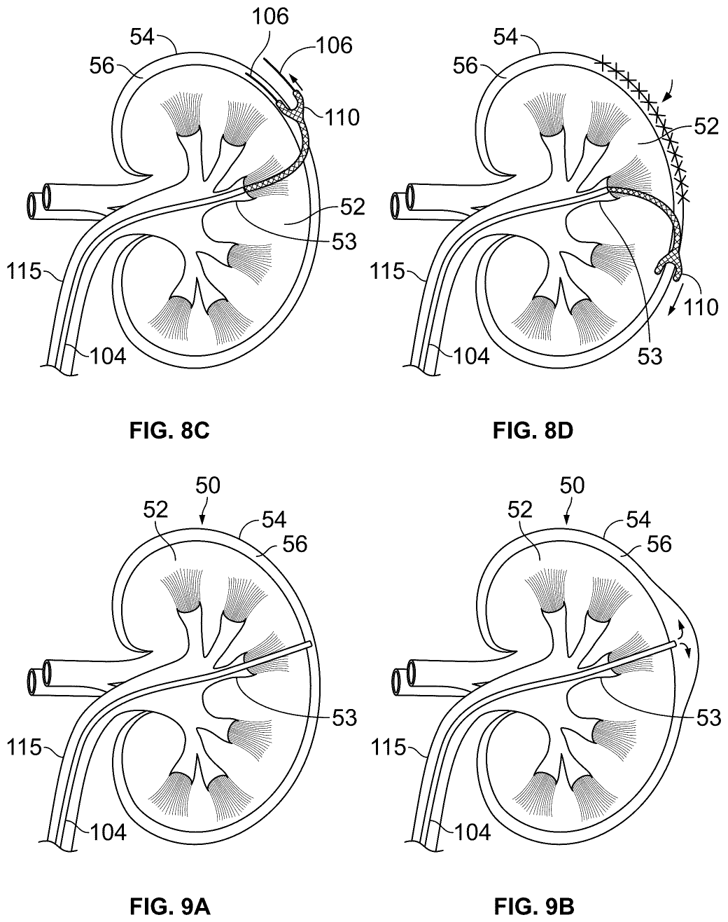

In various treatment procedures (See FIGS. 8A-D), it can be desirable to separate the renal capsule 54 from the parenchyma 52 to thereby decapsulate the target kidney or create more of a space for the interventional procedure. In particular, carefully dissecting or separating the capsule 54 from the parenchyma 52 can better allow for further manipulation such as capsule release. Multiple passes along the capsule 54 with a cutting element 110 can be taken to decapsulate the capsule 54 as required or desired. Various approaches to capsular distension, dissection and/or inflation can be taken.

Here, the subcapsular location 56 is accessed by advancing an interventional device 104 through a ureter 115 and then advancing a wire 106 through the interventional device 104, calyx 53, parenchyma 52 and into subcapsular location 56 (FIG. 8A). In various aspects, the interventional device can be designed to provide sufficient back up support or stiffness to minimize or eliminate the device from backing out while advancing and/or manipulating other complementary wires or devices or apparatus. Next, a second wire 106 is advanced through the interventional device 104, calyx 53, parenchyma 52 and beyond the subcapsular space and into the perirenal space 108 adjacent the capsule 54. The wires 106 are thus placed to provide a platform along which a cutting mechanism 110 can be guided (FIG. 8C). The cutting mechanism 110 can be a mechanical and/or energy-based device configured to dissect the capsule 54 as the cutting mechanism is advance along the capsule 54 (FIG. 8C-D). The wires 106 and/or interventional device 104 can be steerable or otherwise manipulated to direct the cutting mechanism 110 to release different portions of the capsule 54 as desired or required.

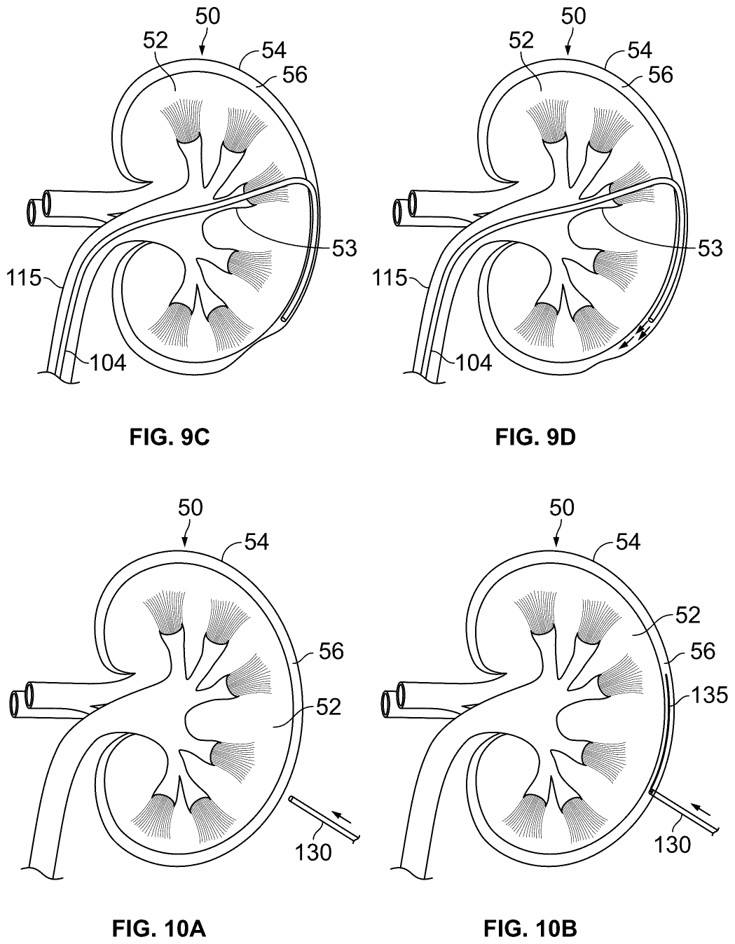

In one approach to intervention (See FIGS. 9A-D), an interventional device 104 in the form of a wire, loop or other catheter 110 can be swept along the capsule 54 to pierce and/or dissect or separate the capsule 54. The device 104 can additionally or alternatively include vibration means to facilitate desired separation or dissection.

That is, an interventional device 104 can be advanced through a ureter 115 and then through the calyx 53, parenchyma 52 and at the subcapsular boundary 56 (FIG. 9A). Once at the subcapsular boundary 56 (FIGS. 9B-D), the interventional device 104 can be steered or manipulated to then be advanced along the subcapsular boundary 56 as desired to accomplish the release or separation of the capsule 54.

In another approach, the subcapsular space can be temporarily filled with fluid or air or other gas to achieve or facilitate desired separation or distension of the capsule 54 from the parenchyma 52. In one approach, a catheter can be placed in the subcapsular location 56 and the fluid or air or other gas introduced. The catheter can be manipulated to directly target sites of adhesion and direct varying forces of jets to achieve separation. Such jets can also be used to perforate or cut the capsule in related treatments.

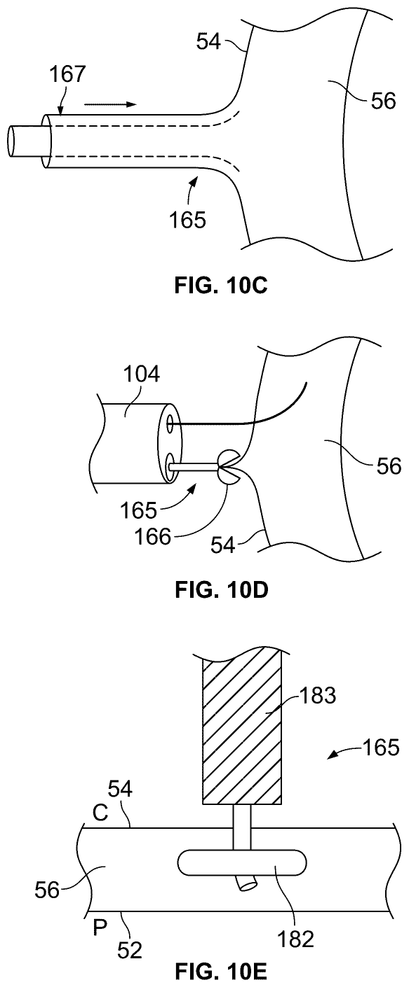

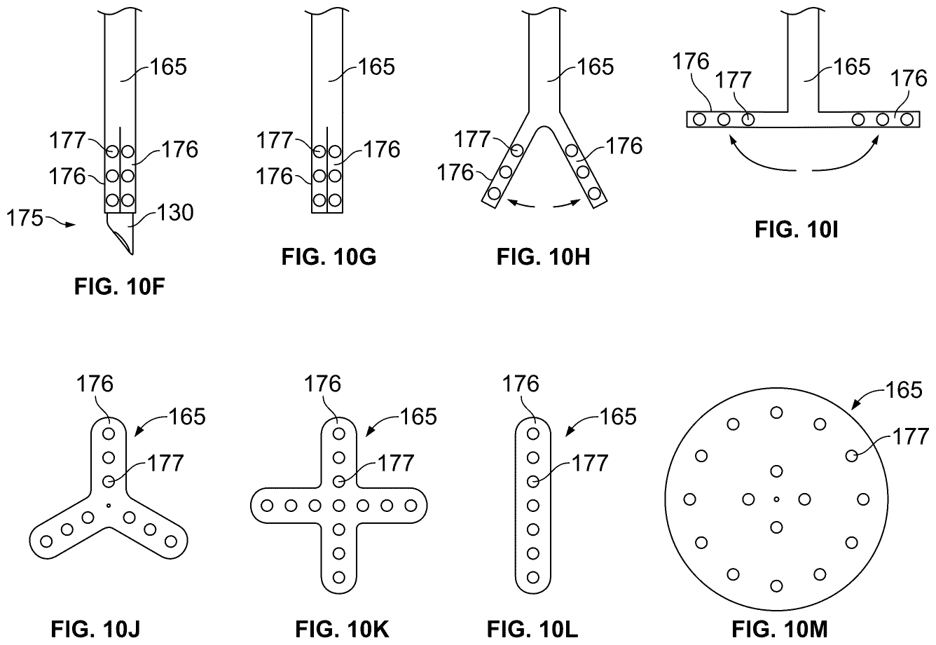

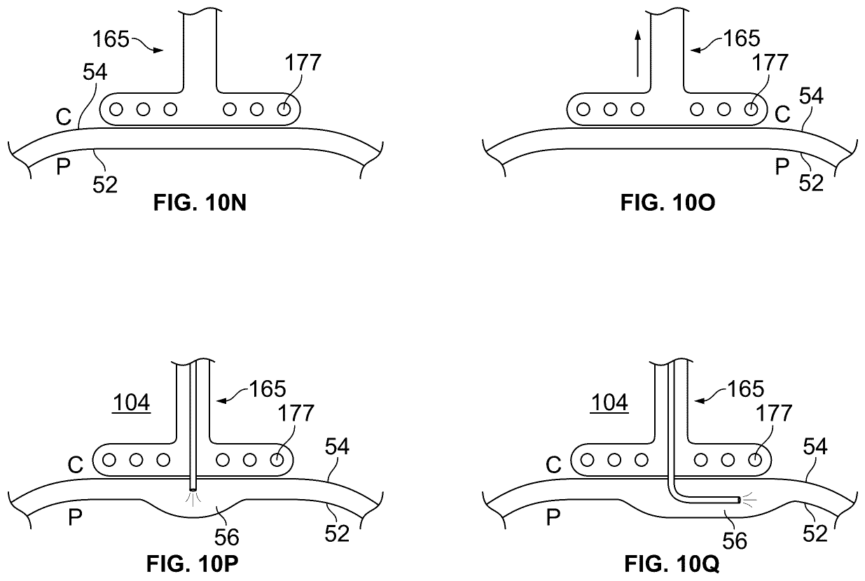

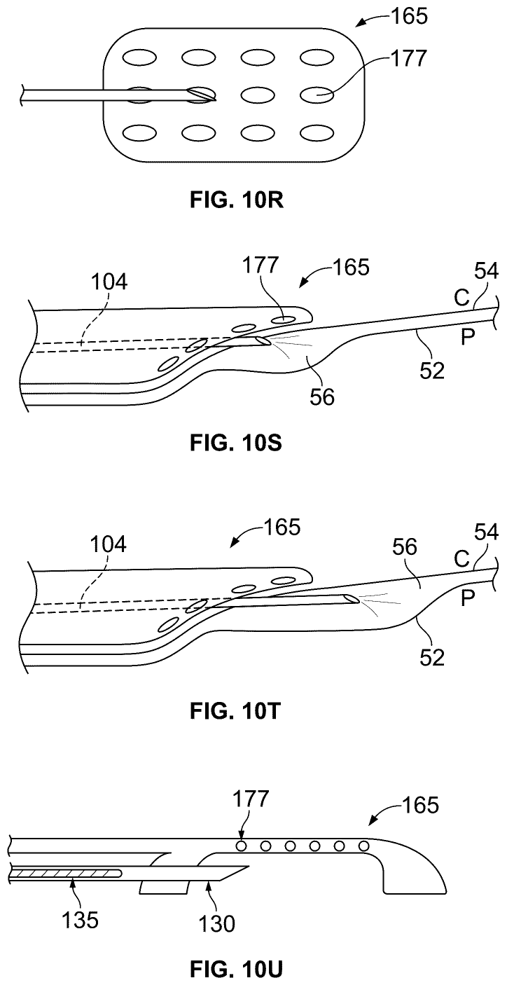

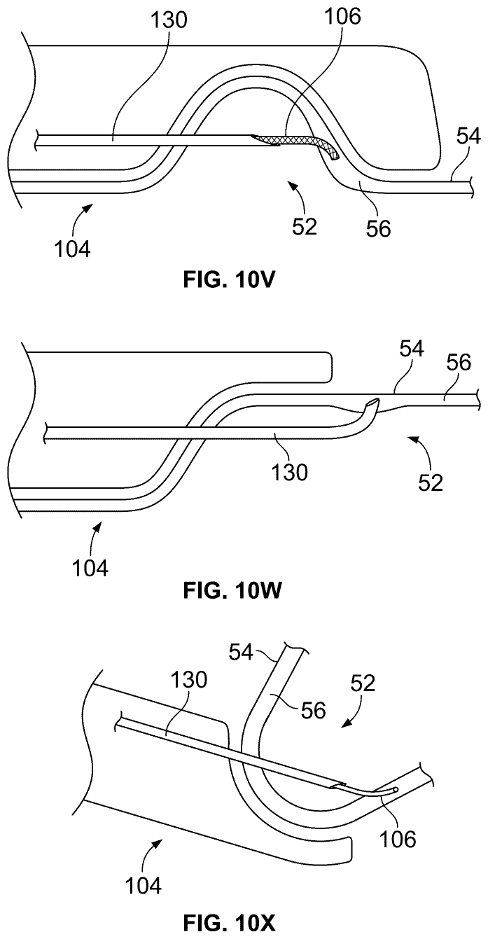

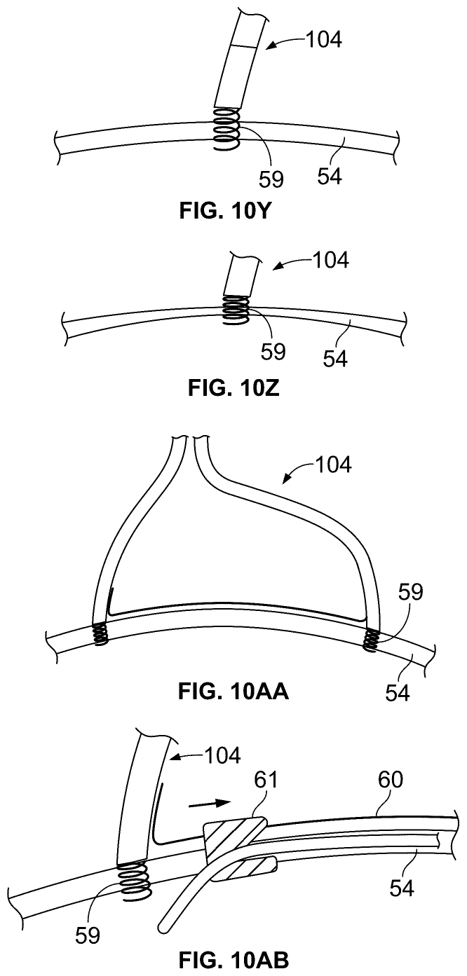

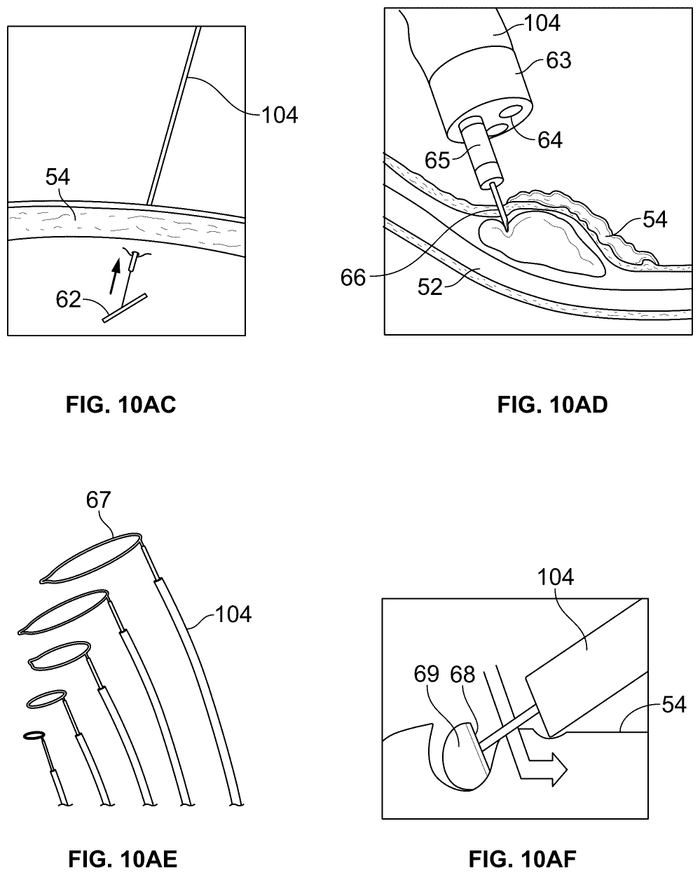

In yet further alternative or additional aspects, a transcutaneous or percutaneous approach can be taken in a renal capsule access and/or pressure reduction treatment procedure (See FIGS. 10A-AF, 11A-B, 12A-B and 13A-B). Again, each of the herein described devices or assemblies can be used or modified in an interventional procedure while taking the transcutaneous or percutaneous approach. Here, however, the interventional site can be reached from an external percutaneous approach via a needle through the ribs to the kidney. The apparatus employed can be a combination of one or more of a needle, catheter or endoscopic based approach guided by fluoroscopy, ultrasound, surgical navigation with or without prior imaging, or a combination of some or all of such modalities.

Further, as stated, cutting of the capsule can be from outside of the capsule and there may not be a need to be within a subcapsular space to accomplish cutting the capsule. By controlling the capsule or by pulling the capsule or adding liquid to dissect the capsule from the parenchyma then cutting the capsule does not involve entering the subcapsular space to allow the parenchyma to drain and/or expand. Further, applying a tension to an outside of the capsule alone can accomplish the desired dissection of the capsule from the parenchyma. Again, dilation of renal tissues is not equated herein with cutting of renal tissues.

In one particular aspect, approaching the interventional site can involve sheath sizes ranging from 4.8 French to 32 French. Since the treatment procedure does not require entering the renal parenchyma to retrieve stones, for example, the system component sizes can be kept relatively smaller so that the intervention is minimally invasive and associated with fewer procedural complications, morbidity and tissue injury. Further, the safest, simplest and/or easiest approach angle to the treatment site can be taken when not focusing on the calyx or stone retrieval. Having such flexibility allows the interventional procedure to be modified and/or repeat procedures as needed knowing that the capsular tissue can be difficult to engage especially when it is unpredictably adhered to the parenchyma.

In one particular aspect (FIGS. 10A-B), capsular release can involve passing a needle 130 across a capsule 54. The passing of the needle 130 across a capsule 54 can be detected by sensing or feeling a pop or change in tissue engagement. Also, a sensor or visualization (i.e. endoscope) can be incorporated into interventional instrumentation to observe this passing or retro-flow of fluid can be indicative of piercing the capsule 54. In one embodiment a safety trocar can be incorporated into interventional instrumentation to retract a sharp tip, so that the piercing of tissue ends with the capsule being pierced and further tissue is not engaged.

With reference to FIG. 10B, it may be desirable to obtain access to the interventional site and then pass a guidewire 135 into the target space. Alternatively, the treatment procedure can also be performed without the use of a guidewire. The needle 130 gaining access can be exchanged and a catheter (not shown) can be passed to secure a subcapsular space. Thus, sufficient access to an interventional site can be achieved just by placement of a wire or sufficient access can be achieved or maintained by additionally grabbing or otherwise controlling the capsule or other tissue with additional or supplemental instrumentation. A pulling action can then be applied to tent the capsule as desired.

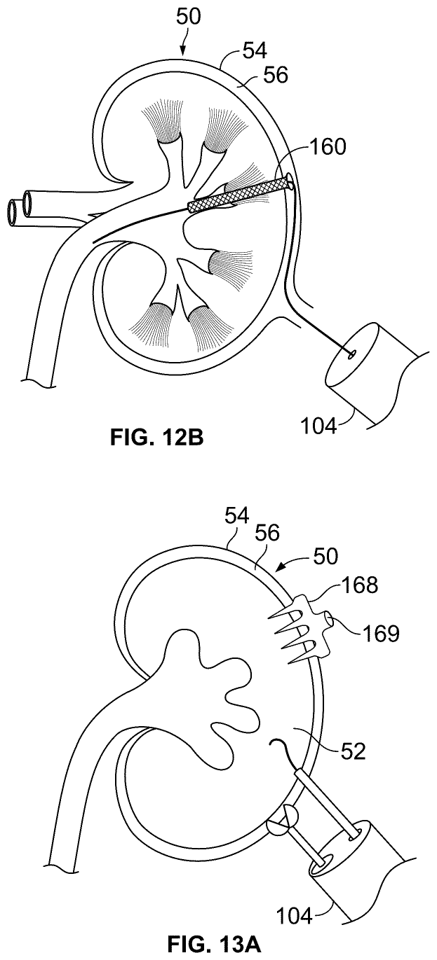

Alternatively, a grasper, suction or other interference mechanism 165 can be used to grasp, twist or engage and control the capsule structure (See also FIGS. 12A-B). In one approach, the grasper or other interference mechanism 165 can embody a nested tube arrangement 167 (FIG. 10C) including an overlapping-tube distal end portion configured to engage renal tissues or can be blunt scissors or tweezers 166 (FIG. 10D).

In one embodiment, one or a multitude of wires, arms or other engaging elements can emerge from a treatment device on the inside of the capsule and flare out, to define structure that can be used to pull the capsule away from the parenchyma below. The wires, arms other engaging elements can project from a distal end or along a length of the treatment device. Those elements may pierce through the capsule on their own or may emerge from the treatment device that has pierced the capsule. In particular, during the deployment of the one or multiple elements, they may pass through the parenchyma to some degree but not pass through the capsule-thus providing an anchoring element to pull or otherwise provide traction on the capsule.

Further, as shown in FIG. 10E, an interference mechanism 165 can include a balloon or other expandable structure 182 that is deployable within a subcapsular space and configured to sandwich the capsule 54 between the balloon 182 and a tube structure 183 from which the balloon 182 is advanced. Such apparatus can additionally be employed for mechanical interference control such that the capsule 54 is pierced and the interference mechanism 165 is placed beyond the capsule 54. Either by expansion or other mechanical interference means, the interference mechanism 165 is pulled back to secure edges of the capsule 54. At all times, securing of the capsule 54 can be confirmed by pulling or tugging and confirming movement of kidney structures via fluoroscopy or ultrasound or the like. Notably, pulling on the secured capsule 54 can facilitate dissection of the capsule 54 from parenchyma 52.

Complete circumferential capture of a capsule 54 can be conducted or only a part of the capsule 54 can be grasped and controlled. In this way, the capsule 54 can be held stationary at a site of entry to thereby stabilize or control for traction. Subsequently deployed instrumentation can be used to create the desired release of tissues.

In one or more of the disclosed approaches, a vacuum can be used to create dissection space from a percutaneous approach. The vacuum can also be used to control or secure with enough force the surface of the capsule. With such control of the capsule tissue, the capsule can then be pierced with a needle or other treatment device, and fluid can be injected to begin the separation of the capsule from parenchyma. Multiple vacuum holes can be provided and employed to best capture capsule tissue such that when an initial hole in the capsule is created and the capsule separates from the parenchyma and becomes free, other vacuum holes can maintain control the capsule. Further, the vacuum source or sources can be configured to be discrete or individual to selected vacuum holes, such that vacuum can be adjusted or turned on and off to different vacuum holes or groups of vacuum holes as needed. In this regard, it may be desirable to turn a vacuum off to certain areas to prevent suction of fluid or to enhance control of certain areas of the capsule such as free edges etc.

With reference to FIGS. 10F-U, a treatment device can include various vacuum interference mechanisms 165 that can be employed to selectively control or secure a capsule. Controlling the capsule allows for counter-traction for capsular incisions as described above, such as for forward cutting actions. In various approaches, the capsule controlling structure can be advanced over a needle or other introducer.

As shown in FIGS. 10F-I, an interference mechanism 165 mounted on a needle 130 can embody a tip 175 that splits to reveal two arms 176 that contain vacuum holes 177. As the arms 176 separate, they can be configured to flatten enough to ride on a kidney wall and engage the capsule with a vacuum. In this regard, the arms 176 can define elastically deformable structure that can be housed within a delivery sheath (not shown) and be configured to be retrievably ejected so that the arms 176 splay outwardly for use and are compressible for withdrawal within the delivery sheath or for transferring to another treatment site. In alternative approaches, the interference mechanism 165 can define various shapes and include three (FIG. 10J) or four (FIG. 10K) arms, define a single elongate arm or two arms if connected at a center to the shaft (FIG. 10L) or a circular base (FIG. 10M).

In use (See FIGS. 10N-Q), the vacuum interference mechanism 165 is placed against a capsule 54 and a vacuum is applied. Once control of the capsule 54 is achieved, an extendable portion of a treatment device 104 is inserted through the interference mechanism 165. The treatment device 104 is then manipulated to dissect tissue such as separate the capsule 54 from the parenchyma as desired.

In an alternative aspect (FIGS. 10R-T), the vacuum interference mechanism 165 can be placed more inline than perpendicular to a capsule 54. In this approach, the interference mechanism 165 applies a vacuum to the capsule 54 and then and extendable portion of the treatment device 104 is advanced more in parallel to a surface of the capsule 54, and is passed to create the subcapsular space.

As shown in FIG. 10U, the inline vacuum interference mechanism 165 can have various configurations. As with each disclosed embodiment, in use, access to a retroperitoneal space is gained. The interference mechanism 165 is then placed on the kidney in a pre-determined location. Suction is applied and an introducer needle 130 is advanced through supporting structure (such as a through hole) provided by the interference mechanism 165, and through the capsule 54. Notably, the location of the needle can be adjusted or placed such that it pierces the capsule 54 and is just inside the capsule wall in-line, so that it is not in-line to pierce the parenchyma or to minimize interaction with the parenchyma, and that needle ‘depth’ could be adjusted as desired. A guidewire 135 is then advanced through the needle 130 and into the subcapsular location 56. Suction can then be released while the wire 135 is continued to be advanced. Next, the interference mechanism 165 can be removed leaving the guidewire 135 as a platform for advancing and manipulating other treatment devices within the target treatment area.

In yet other additional or alternative approaches (See FIGS. 10V-X), it may be desirable to enter the subcapsular location 56 from the extracapsular space and through the parenchyma 52. A treatment device 104 can be employed or configured to pierce the renal capsule 54, and include structure that extends through the parenchyma 54 and then back out of the parenchyma 54, but then not pierce the capsule 54 and stay within the subcapsular location 56. This can be accomplished employing a combination of devices or one device 104 with one or more features that allow this subcapsular access.

In one embodiment, the treatment device can include a needle 130 that is introduced percutaneously and approaches the kidney such that the trajectory of the needle 130 pierces the capsule 54 and then pierces the parenchyma 52 and then exits the parenchyma 52 but stays within the capsule 54. The tract within the parenchyma 52 can be short and shallow, taking a very short path through the parenchyma 52. The needle 130 can stay within the parenchyma 52 and then a catheter or a guidewire or other device 106 may be extended further such at the end exits the parenchyma 52 and positioned within the subcapsular space without piercing through the capsule 54 again and into the extracapsular space on the other side.

The device (needle, catheter with edge capable or piercing capsule and parenchyma) 130 may stop short of exiting the parenchyma 52 and fluid dissection may complete the path to create the subcapsular space. The extended device 106 can use blunt dissection or fluid dissection to travel through the parenchyma 52 and then dissect/travel in the subcapsular space.

The treatment device 104 may be placed against the kidney and suction may pull a section of the kidney (including capsule and parenchyma) into the body of the device to allow the needle 130 to pass through the capsule 54 and parenchyma 52. The device 104 may have a concave face to allow the kidney tissue to conform to the concave face and allow passage of the needle 130 or other device through the capsule 54 and parenchyma 52.

The treatment device 104 can define an interference mechanism (as described above) and can also be shaped and placed such that vacuum is not required to pull the kidney into the device 104. The device 104 may be pushed against the kidney at such an angle that the trajectory of a needle 130 (straight or curved) can pass through the capsule 54 and parenchyma 52 and allow access to the subcapsular location 56. The needle 130 may be made of a shape memory or superelastic material such that it passes through the device 104 and passes through the capsule 54 and parenchyma 52. Again, these approaches may be used with or without suction to position the capsule 52 into the device 104. In one aspect, the treatment device 104 itself may be of sufficient stiffness to provide enough pressure/force against the tissue to allow apposition and puncture into the capsule 54.

To minimize bleeding, the treatment device 104 or its components can be held in place for a period of time to provide pressure or tamponade. As the device 104 is removed, a plug (solid or viscous; not shown) of coagulant or fibrin or the like can be positioned within the parenchymal tract that was formed. Alternatively or additionally, a separate device that is optimized for delivery of pressure can be employed to provide pressure or tamponade against the renal tissues.

In yet another approach to engaging and/or controlling a capsule 54 (See FIGS. 10Y-Z), a treatment device 104 can be equipped with a helix anchor 59. The anchor 59 is configured to cross the capsule 54 and then compress such that the capsule 54 is locked in compression between turns defining the helix anchor 59, thus providing a stable platform connected to the capsule 54. By so connecting the anchor 59 to the capsule 54, a working channel can be provided through a lumen extending through the treatment device 104. Once anchored, the treatment device 104 can also be translated or pulled to manipulate the capsule 54 as desired.

In one specific application, two or more anchors 59 can be attached to the capsule 54 (See FIGS. 10AA-AB) and a flexible member 60 can be configured to span between the two anchors 59. A cutting unit or element 61 can be further provided to be advanced along the flexible member 60 to create a controlled cut in the capsule 54 between the anchors 59. The cutting unit 61 can be partially introduced when the anchor is placed and can include features to lift the capsule to protect against engagement with the cutting unit 61 as it is advanced. Further, the capsule 54 itself can act as a guide such that the cutting element 61 does not dive or deviate from cutting the capsule 54.

As shown in FIG. 10AC, mechanical means can be used to lift targeted tissue. Here, a capsule 54 can be engaged and manipulated or pulled using a treatment device 104 including a T-tag 62 configured to be deployable from a distal end of the treatment device 104. Here, an extracapsular approach to the capsule 54 can be taken and the treatment device 104 can be used to pierce a capsule 54. Once the capsule 54 is by-passed with the treatment device 104, the T-tag 62 can be advanced from within the treatment device 104 and allowed to expand to define a T. Once so configured, the treatment device 104 can be manipulated so that the T-tag 62 engages an inside of the capsule 54 with a desired force or tension, for example, to separate the capsule 54 from parenchyma.

In another aspect, a kidney is approached externally also without use of a guidewire. As shown in FIG. 10AD, a treatment device 104 can include an elongate endoscope 63 sized and shaped to access a capsule 54 and can embody a steering mechanism configured to facilitate navigation to a target site. The endoscope 63 can be equipped with a camera 64 and a working channel through which a longitudinally extending and retractable member 65 can be advanced. The member 65 further includes a lumen through which a needle 66 can be advanced, or fixedly placed.

In use, the needle 66 is employed to introduce a liquid substance beneath a renal capsule 54. This instigates a deliberate separation between the capsule 54 and the parenchyma 52. Subsequent to a successful separation of the capsule 54, specialized devices can be employed to effectuate an incision, enterotomy or analogous interventions.

With reference to FIG. 10AE-AF, various devices can be used to remove or incise a subcapsular layer of a kidney. In one approach (FIG. 10AE), a treatment device 104 can be equipped with a monopolar snare defining a wire loop 67. In use, the wire loop 67 can be used like a noose to capture tissue and then pull the tissue into a bunch. Once the wire loop is expanded and placed on top of and tightened about targeted tissue, an electrical current can be run through the wire loop 67 which operates to cut and cauterize captured tissue. The loop 67 can also be configured to grab the capsule or other target tissue and the loop 67 can be closed or cinched about the targeted tissue to control the tissue without employing cautery energy. If tissue is desired to be cut, cautery energy can then be applied.

In another approach to creating an incision or removing tissue (FIG. 10AF), a treatment device 104 can alternatively or additionally include a knife 68 with an insulated tip 69. The insulated tip 69 and knife 68 are configured and cooperate to present a precise cutting and dissection tool. The insulation 69 helps control the depth of an incision and minimize a risk of unintended damage to surrounding tissues. Energy is passed through the knife 68 that facilitates and allows for cutting and coagulation of targeted tissue.

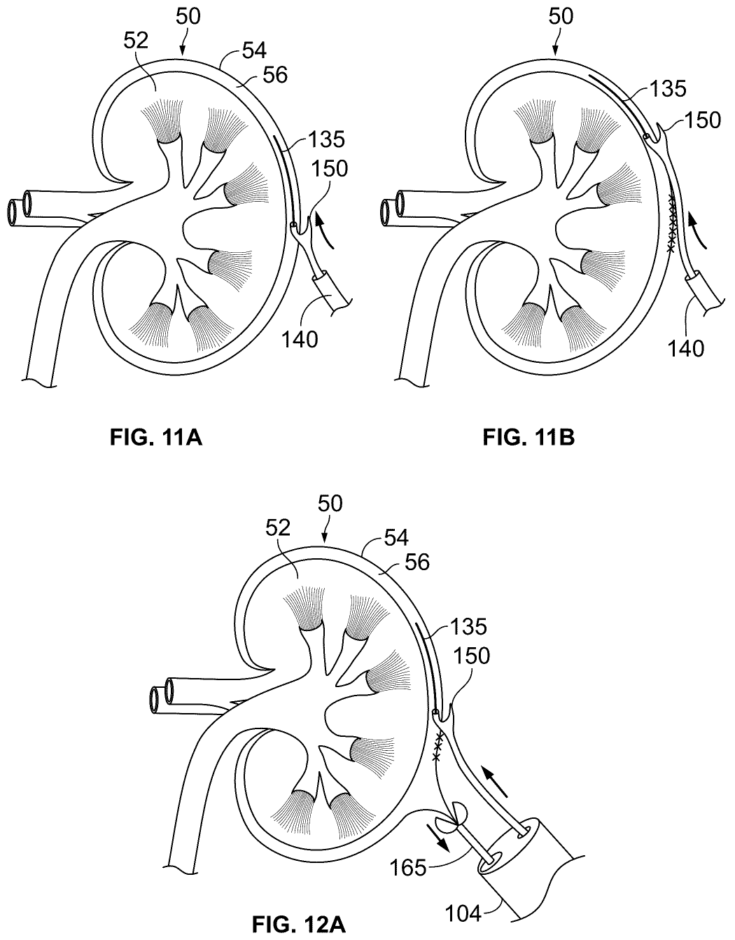

Multiple instruments can be configured to pass through an access site created by the needle 130. The needle 130 can be removed from the site and a working sheath 140 can be placed through which some or all interventional instruments or devices can be passed to minimize trauma to tissue (See FIGS. 11A-B). A wire 135 can be manipulated under the capsule to thereby providing a platform to allow a releasing device 150 to track along it for guidance and safety. Cutting action whether mechanical and/or energy-based approaches, can take place along this path and repeated as necessary or as desired. Multiple passes can be made to create multiple tracts of release and/or fenestration.

Further, with reference to FIGS. 12A-B, a shunt 160 can be placed directly into the kidney at a site adjacent a capsular entry. Alternatively, a shunt 160 can be placed remotely from a capsular entry site so that the capsule 54 above the shunt 160 is intact in case of any adhesion of the capsule 54 at the site of intervention. Such an approach can additionally or alternatively include grasping of the capsule 54 for control purposes and/or releasing the capsule 54 using a cutting mechanism 150 of an interventional device 104. Here, the interventional device 104 would include multiple lumens through which the various implements can be delivered to a treatment site.

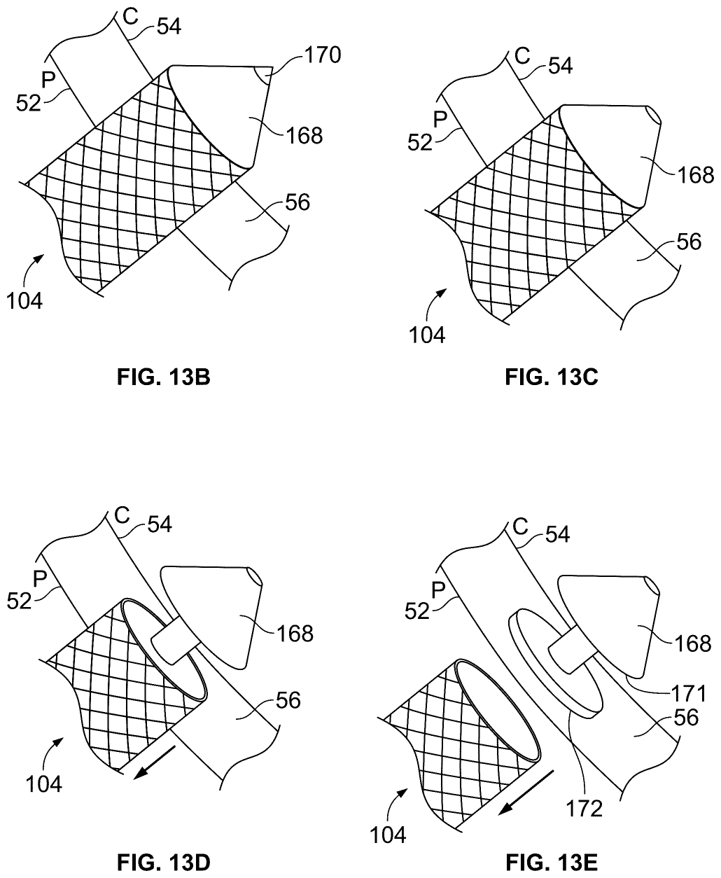

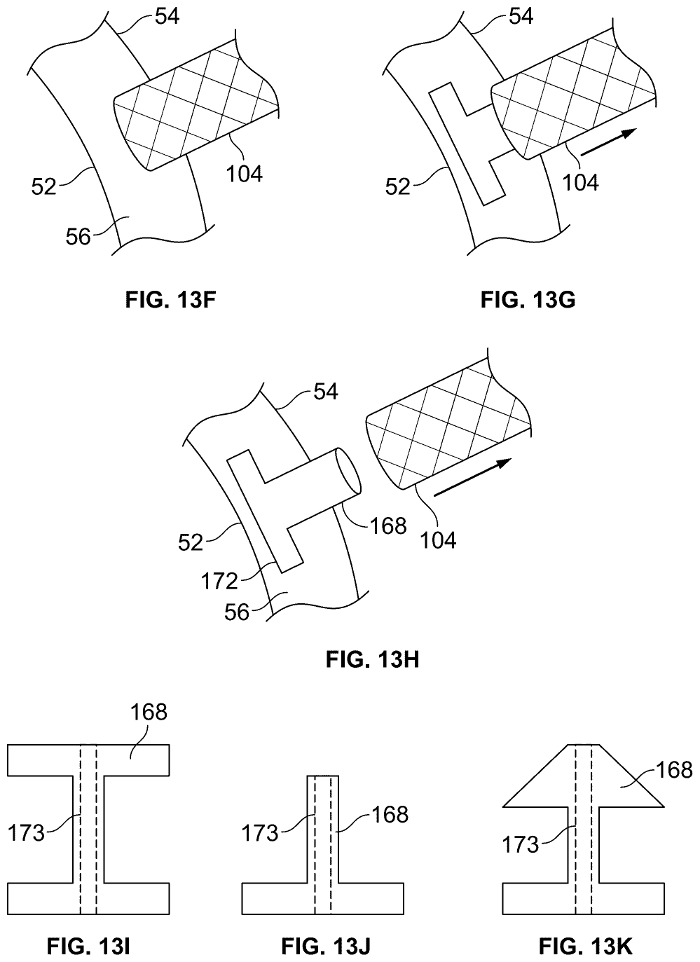

As shown in FIG. 13A, in one or more of the disclosed treatment approaches, a spiked spacer or grommet 168 can be placed within a subcapsular space or otherwise on a capsule 54 solely or in combination with other treatment modalities. The spike grommet 168 includes spikes or other structure configured to retain the grommet 168 in place. The grommet 168 further includes a channel 169 through which fluid can flow away from the kidney to thereby release or reduce renal pressures. The grommet 168 can be placed so that it tents the capsule 54 away from parenchyma tissue 52 to maintain or increase a subcapsular space or release the capsule 54.

In this regard, the spacer or grommet can embody a stent or rivet structure facilitating continuous drainage and alleviating interstitial pressures. The stent or rivet ensures structural integrity while allowing for sustained and controlled release of fluids. A deployment mechanism can be designed to provide secure fixation within the capsule, thereby preventing inadvertent migration. The device aims to establish a regulated drainage pathway, mitigating interstitial pressure and contributing to improved renal function. In a further or alternative aspect, there can be provided a pressure regulating valve, such as a one-way valve to ensure a proper pressure regulation and can additionally be sensorized to monitor the interstitial pressure. An external adapter can be alternatively or additionally be provided to allow a drainage tube or shunt to be connected. Moreover, external or internal surfaces can be coated with a buttress material that will inhibit the healing cascade and ensure the opening stats viable for a prolonged period of time.

In one or more aspects, a suction device or pump (not shown) can be configured on an extracapsular space of one or more spacers to suck fluid through the spacer or push fluid through the space to improve a rate at which fluid is moved from the subcapsular space to the retroperitoneal cavity. The suction device or pump can be an electrically powered apparatus or it can be passively powered by employing a spring-loaded mechanism. Alternatively or additionally, such devices can be configured to push fluid through a tube that extends from the spacer to the skin or from the spacer to an opening created in the bladder in which a tip of the tube is sewn or otherwise attached.

In other approaches (See FIGS. 13B-K), spacers or capsular grommets can be placed within a subcapsular space for draining purposes, without the use of a guidewire. In one approach, a treatment device 104 can include a piercing terminal end defined by a retractable point 170 and a grommet 168 (FIG. 13B). The assembly can be advanced through the parenchyma 52 and capsule 54. Once through the capsule 54, the point 170 is retracted (FIG. 13C) and then the device 104 is withdrawn leaving the grommet 168 in place across the capsule 54 (FIG. 13D). Alternatively or additionally, the point 170 can be automatically withdrawn upon sensing the piercing of the capsule 54 so that further tissue is not engaged and is provided as a safety mechanism. Further, the treatment device 104 can include a pusher or stopper element (not shown) that accomplishes a controlled ejection of the grommet 168 from the treatment device 104.

In one aspect, no further dilation of the capsule tissue 54 is required. The grommet 168 is configured with a first surface engaging surface or flange 171 configured to reside on an outside of the capsule 54 and a spaced second surface or flange 172 configured to reside within the subcapsular location 56 (FIG. 13E). A through hole extends through the grommet 168 to provide a path for fluid to flow from within the subcapsular space to outside the capsule 54. The spacer or grommet 168 can be formed from a polymer such as silicone or an expandable metal such as nitinol.

In another embodiment, the spacer or grommet 168 is placed using an approach from outside of the capsule 54 (See FIGS. 13F-H). Here, the treatment device 104 is used to puncture or otherwise pass through the capsule (FIG. 13F) and then is withdrawn to release the grommet (FIG. 13G). The treatment device 104 is then completely removed from the capsule 54 leaving the grommet 168 in place across the capsule 54. The grommet includes a portion or section 174 that is configured to reside within the subcapsular location 56 (FIG. 13H). Such a portion 174 can be elastically deformable so that it assumes a reduced profile when housed within the treatment device 104 and then expands once delivered within the subcapsular location 56. In this way, the grommet 168 can be retained in place. The grommet 168 also includes a through hole 173 (See FIGS. 13I-K) that extends through the grommet 168 to provide a path for fluid to flow from within the subcapsular space to outside the capsule 54. As shown in FIGS. 13I-K, the grommet 168 can have various alternative cross-sectional profiles.

As shown in FIGS. 14A-D, capsular fenestration or dissection can involve an interventional device 104 including mechanical blunt dissectors such as a wire 112 or a wire loop 113, or other herein-described mechanisms can be employed additionally or alternatively. The interventional device 104 is manipulated to engage or be placed adjacent the target kidney 50 (FIG. 14A). Next, a wire or other cutting device 104 is advanced through the interventional device 104 and manipulated to engage the capsule 54 surrounding the kidney 50. Multiple passes or varying lengths and angles along the capsule 54 are taken to decapsulate the kidney 50 as desired or required (FIG. 14B). Such passes can be a result of pushing or pulling action of the cutting device 104 along target tissue. Subsequently, the interventional device 104 is removed from the treatment site.

In a related approach (FIGS. 14C-D), a looped wire 112 can be passed through a previously placed interventional device 104. As the loop 113 is deployed from a distal end of the interventional device, the loop 113 decapsulates the kidney 50 as desired. The interventional device 104 and loop 113 can be manipulated and translated to treat various targets of the kidney 50 and then is removed from the treatment site when the procedure is deemed completed.

In one specific approach, the interventional device can include a pair of pre-formed looped wires 112 or other atraumatic elements that can be manipulated to separate the capsule 52 (See FIG. 14E-F). The use of loops would allow the shape to be advanced atraumatically either creating separations or allowing anchoring to the capsule 54 (See also FIGS. 14G-H).

With reference to FIGS. 14I-M, multiple paths of dissection and capsular release can be undertaken. From one or multiple sites of entry through a capsule 54, the treatment device 104 can be advanced along various paths to achieve dissection goals. The treatment device 104 can embody any of the disclosed structures including catheters supplying pressurized fluid or wires, for example. In one approach, the treatment device 104 is advanced and employs fluid to accomplish dissection up an anterior side of the kidney from the posterior pole. From the same location, the device 104 is sent along a posterior side of the kidney thereby achieving dissection spaces on both sides of the kidney.

In one or more approaches, it may be desirable to target a portion of the kidney for entry to allow easier access to both an anterior and posterior sides of the kidney. Multiple tracks can be made to create lines of capsular release or cutting or disruption. Moreover, various patterns of cuts or punctures can be formed in a capsule 54 to provide the capsule 54 with desired flexibility and/or an ability to expand (See FIGS. 14N-P). In this way, the so treated kidney can be adapted to better respond to increased internal pressures because the material property of the capsule 54 itself has been modified to accommodate such pressures. In one approach, the capsule 54 can be cut using a fractional ablative laser that quickly creates full thickness reliefs in the capsule 54. Alternatively, a reciprocating blade or punch can be employed such as in a sewing machine style apparatus.

As shown in FIGS. 14Q-T, a capsule cutting tool 177 can employ a safety mechanism that is configured to protect renal anatomy while a capsule is being cut. In one approach, the cutting tool 177 can embody a step change in diameter from a sharpened point 178 to a shoulder 179. The sharpened point can be configured to have a length equaling a thickness of a capsule 54 so that upon piercing the capsule 54, the shoulder 179 prohibits the point 178 for passing further into renal tissue beyond the capsule 54. In another approach, the cutting tool 177 can include a retracting or spring-loaded tip 178 that retracts within a trocar 181 once the capsule is pierced and a drop in force is detected. Alternatively, the tool 177 can be configured such that the trocar advances over the tip 178 once the capsule 54 is pierced. In these latter approaches, the tool 177 can be equipped with one or more of force gauges and/or be driven electronically (motor, solenoid, etc.) to a safe location once a force change is sensed.

In certain treatment procedures, it can be desirable to employ approaches that minimize unwanted perforations, provide control of direction of entry and/or minimize bleeding in renal tissue access. One or more approaches can involve hollow devices such as needles and catheters that can assist in confirming anatomy, use hydro-dissection as a means for advancement, and/or to create a dissection plane from which to operate other instruments or place an implant or measuring devices. Ultrasound or optical coherence tomography can be used to determine a least vascular zone for placement of a treatment device. Sensors can also be employed additionally or alternatively to aid in navigation.

In one approach, the natural anatomic features of a capsule can be utilized in an interventional procedure. Imaging agents such as liquid radiographic contrast or gas such as carbon dioxide can be used to determine and confirm a location of an advancing device, and/or create a dissection plane that minimizes bleeding. It is also possible to dissect a capsule from other renal tissue to allow for easier and/or safer manipulation, resection, or perforation of the capsule. When desired, a penetrating device can be left in place, either partially or in its entirety, to act as a drainage shunt, means of delivering therapy or as a measuring device.

In various approaches, it can also be desired to use relatively smaller profile microcatheter as using the same can avoid the deformation of anatomy associated with the use of larger profile devices and can be navigated more easily within tissue without the use of a guidewire, for example. The microcatheter can be advanced from a needle or another catheter and can be deflectable to help with navigation at a treatment site. The leading tip of the microcatheter can be sharpened to facilitate advancement and/or can include radiopaque characteristics for locating under fluoroscopy and a length or portion of a length of the microcatheter can be porous or include holes for assisting in draining fluid. In one specific embodiment, the tip can embody material that appears darker than contrast material so that viewing is possible when contrast is used.

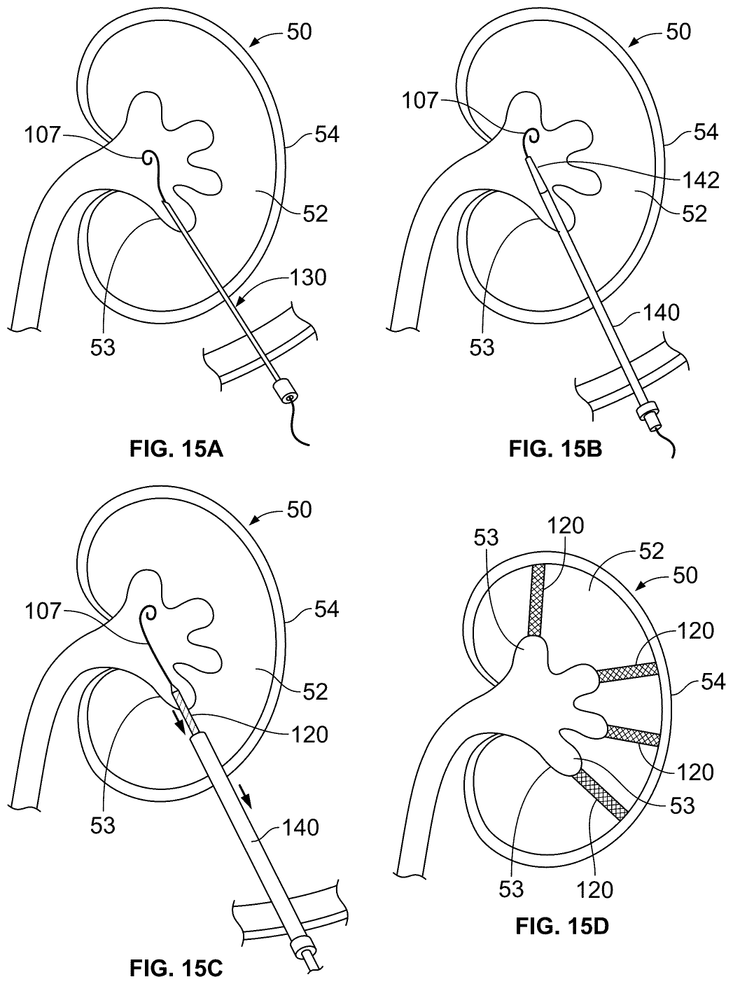

With reference now to FIGS. 15A-D, there is shown an interventional system which can be used to deliver a custom length shunt, stent or other implant 120 through a percutaneously directed access with the aid of fluoroscopy or ultrasound. Here, a Seldinger technique is employed involving a dilator and a guide placement directly into the calyx 53 then then subsequent delivery of one or more stents or implants 120 into place. Again, access to renal anatomy can be along Brodel's line to minimize bleeding. Further, the implant 120 can have side holes or porosity to allow for fluid to enter along its length.

In a first step (FIG. 15A), a needle 130 is advanced percutaneously through the patient's skin and into an interior of a kidney 50. As shown, a terminal end of the needle 130 is positioned within a calyx 53. A wire 107 is next advanced through the needle 130 and placed within a major calyx 53. Alternatively, the procedure can also be performed without the use of a wire.

The needle 130 then can be removed and a working sheath 140 including a dilator 142 configured at its distal end is advanced over the wire 107 to within the targeted calyx 53 (FIG. 15B). The dilator 142 can be expanded or otherwise employed to create a space for the implantation of a stent or other implant 120.

The sheath 140 is then withdrawn to expose the stent or implant 120 (FIG. 15C). As shown, the stent or implant 120 is self-expanding so that upon the withdrawal of the sheath the stent or implant 120 self-deploys in the targeted space within the calyx 53 and across the parenchyma 52 to the capsule 54. Alternatively, the stent or implant 120 can be expanded by a balloon to deliver the structure into place as desired. The delivery sheath 140 is then removed from the interventional site or repositioned for the implantation of additional devices. This procedure can be repeated as necessary to provide multiple stented areas from a calyx 53 to the capsule 54 (See FIG. 15D) to thereby facilitate interstitial pressure reduction of fluids.

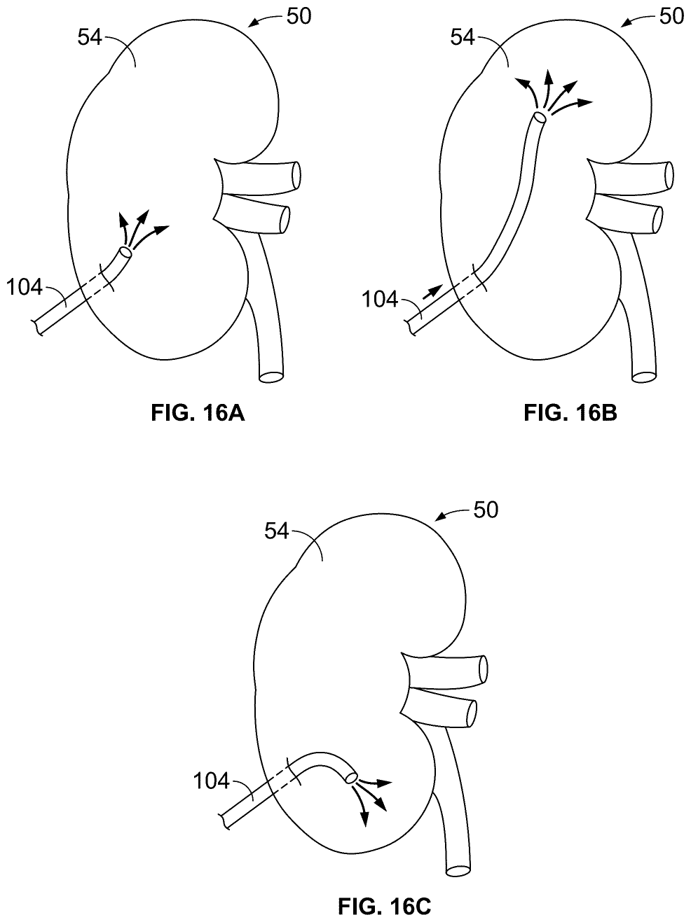

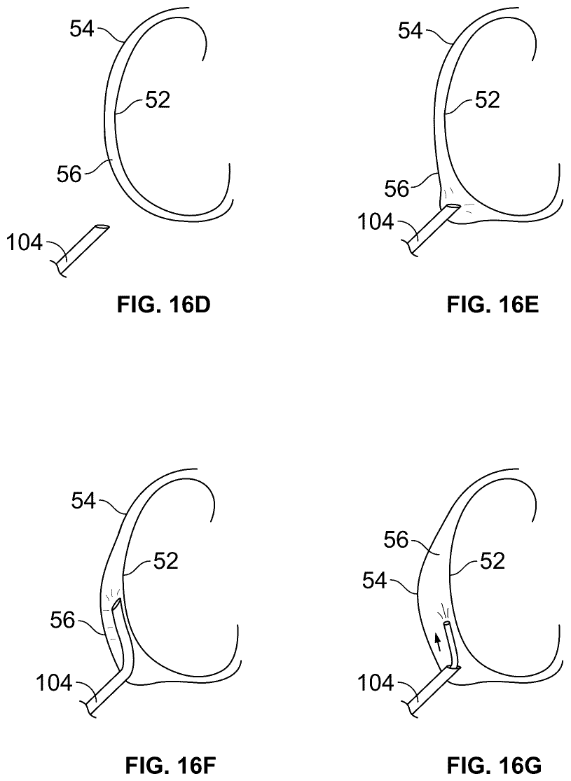

Also, additionally or alternatively, the subcapsular space can be filled with fluid or a gas to distend the capsule 54 from parenchyma 52 to make it easier to secure and control the capsule, or to release the capsule 54. As shown in FIGS. 16A-C, the interventional working sheath or catheter or device 104 includes structure that can be manipulated or steered to thereby provide access to target portions of a capsule 54 through a single-entry point.

Referring now to FIGS. 16D-G, as stated, piercing the capsule 54 from outside the capsule 54 can involve a percutaneous approach. In these and each of the disclosed embodiments, visualization and guidance of a treatment device 104, can be provided via fluoroscopy, ultrasound, CT and/or endoscopic visualization. Also, surgical navigation, OCT, electromagnetic guidance, or the like can be combined or used independently, as needed. In one aspect, when accessing the kidney via a percutaneous approach, it may be desirable to target the posterolateral aspect of the kidney. In a further or alternative aspect, the treatment device 104 can be passed through the ribs and into the kidney aiming for a posterior calyx 53, or the superior pole of the kidney 50.

The treatment device 104 may be connected to a fluid supply so that once the device pierces the capsule 54, fluid from the device 104 can dissect the capsule 54 from the parenchyma 52. The angle of the device 104 approach can be directly at the kidney capsule 54 or directed in a more shallow manner to encourage less penetration into the parenchyma 52 while maximizing fluid flow into the space between the parenchyma 52 and capsule 54 to facilitate dissection. In one or more approaches, is can be desirable to pierce into the parenchyma and then slowly draw the needle back and allow fluid to dissect the space (FIG. 16G).

In various approaches, sensors (not shown) can be placed or positioned to monitor fluid pressure and to facilitate determining spaces or renal anatomy that fluid is present or is entering based on pressure readings. Such monitoring can be combined with positional information data collection provided by surgical navigation or other devices or apparatus.

In one particular aspect, sensors can be placed in parenchymal tissue to sense one or more of physiological parameters in renal parenchyma. The sensors can be configured at a tip of a treatment device or a wire that is advanced to the parenchyma from within kidney anatomy or from the outside of the kidney. The sensors can be placed within the Brodel's plane to take advantage of the avascular anatomy present. Thus, renal parenchymal pressure and other parameters independent of the capsule can be monitored, such as when the capsule is released or a spacer is placed across the capsule.

Further, a contrast agent can be inserted so that a fluid path can be observed via fluoroscopy, such as when fluid flows into the parenchyma 52 versus a subcapsular space, for example. Contrast agents can be used both internal of renal anatomy as well as in the treatment device 104 itself to see what space the treatment device 104 has entered and to help direct dissection of the capsule 54 from the parenchyma 52.

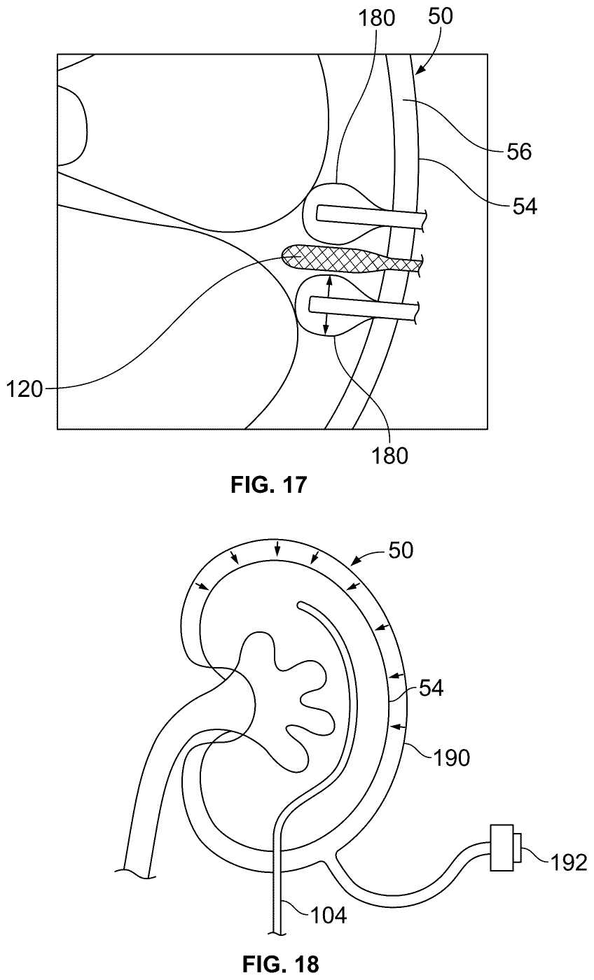

Turning to FIG. 17, there is shown an approach to pump fluids from renal tissue. As shown, a pair of spaced balloon catheters 180 are positioned within a parenchyma 52 on opposite sides of a stent or shunt 120. The balloon catheters 180 can extend to exterior of a patient's body and the shunt 120 can extend from the parenchyma 52 to a perirenal space or also exterior to the body. The balloon catheters 180 are configured to be alternatively expanded and deflated to facilitate the pumping of fluids from the parenchyma 52 through the shunt 120 to thereby reduce renal pressures. These structures can be temporarily or permanently places about a target kidney 50.

With reference to FIG. 18, various approaches to gently massaging or compressing a kidney 50 to ultimately reduce excess fluids from the kidney 50 and reduce renal pressures is shown. In one approach, a jacket 190 can be configured about a target kidney 50. The jacket 190 can include inflatable pockets or other structures that can be activated to cyclically or periodically apply pressures to the kidney 50. In an alternative approach, a highly elastic balloon (not shown) can be placed within a subcapsular location 56 and cyclically or periodically expanded and deflated to apply gentle pressures to the kidney while using the capsule as a base against which applied pressures can be supported. In this way, excess fluids can be caused to be removed from the kidney 50 such as through an interventional device 104 or a placed shunt. These devices can be remotely controlled or they can be directly attached to a pump 192 that facilitates the actuation of the balloon or expandable members or structures.

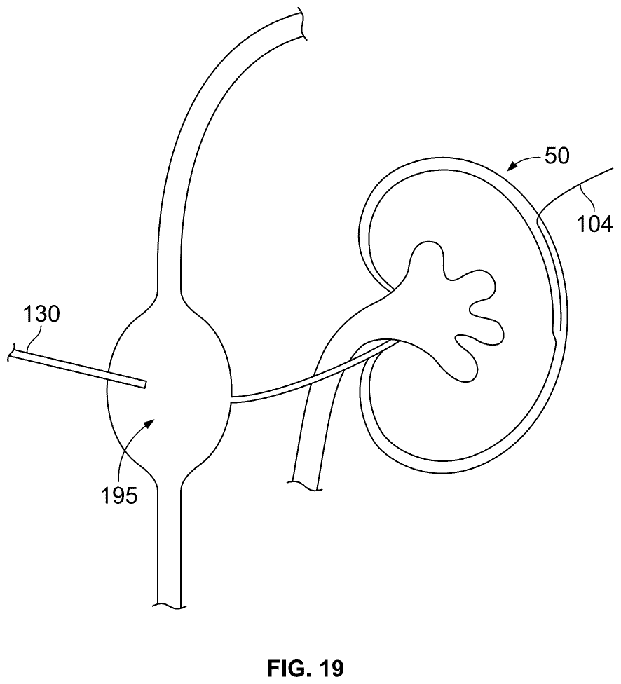

Additionally, as shown in FIG. 19, in another embodiment, a needle 130 can be employed to withdrawn fluids directly from a cisterna chyli 195 to facilitate reducing renal pressures. A percutaneous or other approach to advancing the needle can be taken to engage the cisterna chyli 195. Alternatively, a catheter can be advanced through the ureter or other vasculature to engage and remove fluids from the cisterna chyli 195. Apparatus can include suction via the catheter in the cisterna chyli to facilitate the removal of interstitial fluids and lymph from the kidney and reduce renal pressures. Further, the apparatus can include a filter or means to separate lymph from the fluid and re-insert the lymph back into the patient's system.

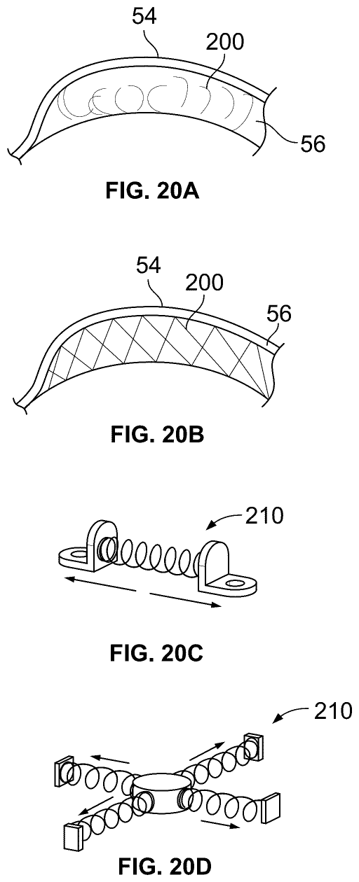

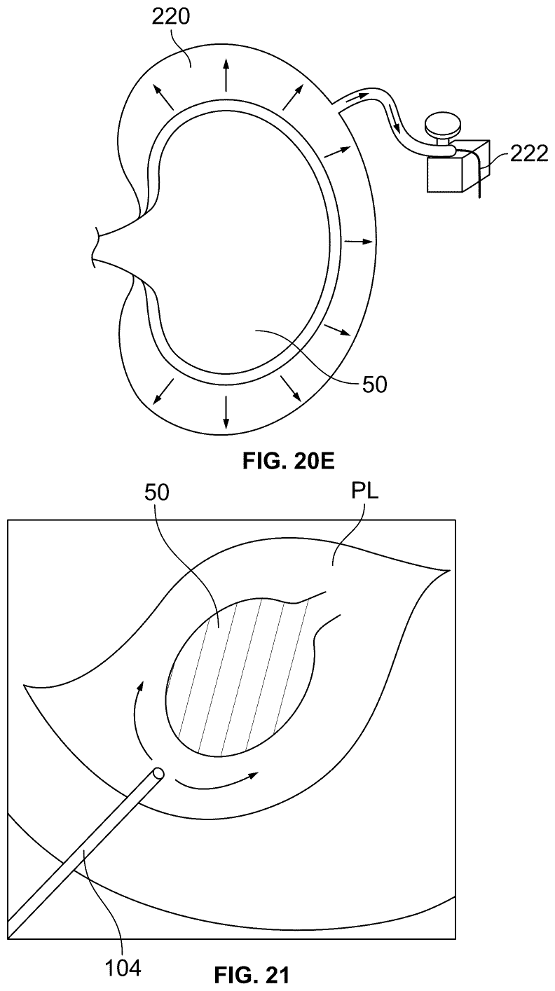

It has been acknowledged that re-encapsulation of the kidney can occur after an injury or a treatment to the kidney involving capsule cutting or dissection or removal. In various aspects, such re-encapsulation can be modified or controlled so that the newly formed capsule could be less restrictive to the kidney. In one or more approaches (See FIGS. 20A-B), a bioabsorbable scaffold 200 can be configured about renal anatomy or within a remaining portion of a capsule 54. The scaffold 200 can remain with the kidney during re-encapsulation and then be absorbed subsequent to the formation of the new capsule, thereby leaving more space for the kidney. A compliant or flexible scaffold can be used so that the scaffold does not itself increase pressure on the renal anatomy as pressures within the kidney changes.

Additionally or alternatively, one or more compression spring devices 210 (FIGS. 20C-D) can be placed or otherwise anchored about a capsule to put the capsule under tension. The spring device 210 can define a linear structure (FIG. 20C). As shown in FIG. 20C, a plurality of springs also can be included in the spring device 210. Notably, such springs can be arranged in various configurations, such as to be directed outwardly from a center point, or they can be arranged in other configurations that facilitate the desired effect. In this way, expanded capsule tissue growth is encouraged, resulting in a more loosely formed capsule about other renal anatomy.

As shown in FIG. 20E, an entirety of a kidney 50 can alternatively or additionally be surrounded with a vacuum chamber 220 that functions to expand the capsule 54 out into the vacuum space created and away from other renal anatomy. Here, in one approach, vacuum is provided to the chamber 220 by a pump 222.

While accessing the renal space, it may be desirable to utilize the same instruments to access the perirenal space. That space is defined by a fascial layer that surrounds the kidney. Removing adipose tissue and other perirenal structures may reduce the perirenal pressure that contributes to increasing parenchymal pressure. Reducing perirenal pressure may also reduce pressure on the renal vasculature and structures located in and near the renal sinus, such as the renal vein and renal artery.

By reducing volume in the perirenal space, there is less volume to compress upon the parenchyma and increase parenchymal pressure. As shown in FIG. 21, the treatment devices or catheters 104 can navigate to various locations within the perirenal space (PS) about a kidney 50, and selectively remove adipose tissue and the like as desired. Mechanisms to remove, shrink, vaporize the desired tissue or structures include suction, vibration to break up/loosen tissue, mechanical removal, energy (laser, rf, heat, cold, ultrasound, etc) to remove/destroy/vaporize tissue and the like.

In various approaches, an interventional procedure can involve a trial period where a catheter drain or other interventional treatment devices are inserted in a kidney for a period of time and the interstitial fluid that is directed to be removed by the drain or other devices is externally collected in order to test for the efficacy of the system. Fluid flow rate is assessed and evaluated using various other clinical parameters prior to the insertion of permanent draining structures.

Further, cauterization or sealing of vasculature or tissues can be conducted in connection with any one of the disclosed approaches to address any damage that is created during or after an interventional procedure. Moreover, in any of the disclosed approaches, devices or means can be provided to prevent an encapsulation of system drainage structures to maintain continuous fluid flow without a barrier being constructed by the patient's body. This may include the use of drug elution to inhibit the formation of endothelium or connective tissue which might create a barrier to continue fluid flow.

In one or more of the described procedures, local anesthesia can be used. Additionally, as stated, various imaging modalities can be employed in connection with one or more of the above-described interventional procedures. A combination of fluoroscopy, ultrasound, direct visualization, MRI or CT scanning, and/or surgical navigation can be utilized to facilitate the intervention. Catheter or other interventional instrumentation can embody intravascular or other ultrasound generating structures, OCT, endoscopy for visualization, or navigation functionality.

It can also be desirable to place biologics, spacers or gels to prevent the formation of new capsule tissue around the kidney. Further, as stated, repeat procedures can be conducted to prevent regrowth or adhesion of the capsule that leads to renal pressure increases. That is, the treatment sites can be moved to new or additional locations to re-release or fenestrate a capsule to thereby reduce pressures. Moreover, as stated, the various approaches to intervention can be combined. For example, a ureteral approach to access a subcapsular space can be combined with sending a guidewire through a capsule, and a percutaneous approach can be taken to capture the guidewire from the ureter to more easily capture the capsule. In each of the disclosed approaches to treatment, a particular treatment procedure can also be performed without the use of a guidewire.

Accordingly, various approaches for renal access and pressure reduction are presented. The disclosed approaches are configured to provide an effective and focused approach to provide renal capsule access and pressure reduction. The various approaches of the present invention provide methods for treating Heart Failure (HF) and other cardiac and kidney related conditions such as Acute Decompensated Heart Failure (ADHF), Cardiorenal Syndrome (CRS), Acute Kidney Injury (AKI), Chronic Kidney Disease (CKD), Hypertension and Diabetes.

While the present disclosure has been described with reference to the specific embodiments thereof, it should be understood by those skilled in the art that various changes may be made and equivalents may be substituted without departing from the true spirit and scope of the disclosure. In addition, many modifications may be made to adapt a particular situation, material, composition of matter, process, process step or steps, to the objective, spirit and scope of the present disclosure. All such modifications are intended to be within the scope of the present disclosure.

That which is claimed is:

1. A system for renal access and pressure reduction, comprising:
an apparatus that provides access a kidney; and
an interventional apparatus that engages a renal tissue, the interventional apparatus being sized and shaped to be advanced to the kidney.

2. The system of claim 1, further comprising a needle having a distal end configured to pierce a renal capsule.

3. The system of claim 1, further comprising a catheter or sheath sized and shaped to provide a path for the interventional apparatus configured to engage the renal tissue.

4. The system of claim 1, further comprising a guidewire sized and shaped to be advanced to engage the renal tissue.

5. The system of claim 1, wherein the interventional apparatus is sized and shaped to be advanced within vasculature to the kidney.

6. The system of claim 1, wherein the interventional apparatus is sized and shaped to be advanced percutaneously to the kidney.

7. The system of claim 1, wherein the interventional apparatus is sized and shaped to be advance ureterally to the kidney.

8. The system of claim 1, the interventional apparatus further comprising a grasper configured to engage a renal capsule.

9. The system of claim 1, the interventional apparatus including a cutting element configured to score, fenestrate or pierce a renal capsule.

10. The system of claim 1, the interventional apparatus including grommet configured to be placed within a hole formed in a renal capsule.

11. The system of claim 1, the grommet further comprising spikes or barbs for engaging renal tissue.

12. The system of claim 1, wherein the grommet is sized and shaped to tent a renal capsule away from a parenchyma.

13. The system of claim 1, further comprising a balloon sized and shaped to be placed within a subcapsular location and be expanded to separate a capsule from a parenchyma.

14. The system of claim 1, further comprising a stent sized and shaped to be placed within renal tissue and extend from a parenchyma to a subcapsular space.

15. The system of claim 1, further comprising a stent sized and shaped to extend from a parenchyma to an extra-renal space.

16. The system of claim 1, the interventional apparatus further comprising a loop configured to decapsulate a renal capsule.

17. The system of claim 1, wherein the interventional apparatus is steerable.

18. The system of claim 1, the interventional apparatus further comprising an energy means configured to cut, fenestrate or pierce renal tissue.

19. The system of claim 1, wherein the interventional apparatus is configured to treat multiple areas of a kidney.

20. The system of claim 1, further comprising a jacket configured to massage or compress a kidney.
